(19) United States
(12) Patent Application Publication
(10) Pub. No.: US 2005/0137135 A1
(43) Pub. Date: Jun. 23, 2005

(54) Novel analogues of glucose-dependent insulinotropic polypeptide

(76) Inventors: Simon A. Hinke, Brussels (BE); Susanne Manhart, Halle/Saale (DE); Jan A. Ehses, Vancouver (CA); Christopher H.S. McIntosh, Vancouver (CA); Hans-Ulrich Demuth, Halle/Saale (DE); Raymond A. Pederson, Vancouver (CA)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 11/042,562

(22) Filed: Jan. 25, 2005

Related U.S. Application Data
(63) Continuation of application No. 10/397,160, filed on Mar. 26, 2003, now abandoned.
(60) Provisional application No. 60/368,197, filed on Mar. 28, 2002.

(51) Int. Cl. A61K 38/10; C07K 7/08; A61K 38/14

(52) U.S. Cl. 514/14; 530/327; 514/8; 530/322

(57) ABSTRACT

The present invention relates to novel C-terminal truncated fragments and novel N-terminal modified analogues of gastric inhibitory polypeptide as well as various GIP analogues with a reduced peptide bond or alterations of the amino acids close to the dipeptidyl peptidase IV (DPIV)-specific cleavage site providing improved DPIV-resistance and prolonged half-life. Further the invention relates to novel analogs with different linkers between potential receptor binding sites of GIP. The compounds of the present invention and their pharmaceutically acceptable salts are useful in treating GIP-receptor mediated conditions, such as non-insulin dependent diabetes mellitus and obesity.

### CROSS REFERENCE TO RELATED APPLICATION

This application claims benefit from U.S. provisional application Ser. No. 60/368,197 filed on Mar. 28, 2002, which is incorporated herein by reference in its entirety.

### FIELD OF THE INVENTION

The present invention relates to the area of novel analogues of Glucose-dependent Insulinotropic Polypeptide (GIP), pharmaceutical compositions containing said compounds, and the use of said compounds as GIP-receptor agonists or antagonists for the treatment of GIP-receptor mediated conditions.

### BACKGROUND ART

The incretin GIP (glucose-dependent insulinotropic polypeptide), a 42 amino acid peptide, is released from the K-cells of the small intestine into the blood in response to oral nutrient ingestion. GIP inhibits the secretion of gastric acid and promotes the release of insulin from pancreatic islet cells [1,2]. It has been shown that the combined effects of GIP and glucagon-like peptide-1₇₋₃₆ (tGLP-1) are sufficient to explain the full incretin effect of the entero-insular axis [3]. GIP and the related hormone, tGLP-1, have been considered to be involved in the pathogenesis of type II (non-insulin dependent) diabetes mellitus. The physiological actions of the incretins, and especially of GLP-1, are not only manifested by enhanced insulin secretion but also by inhibition of gastric emptying [4] and suppression of glucagon release [5,6,7,8] and may result in an improved glucose tolerance. Additionally, GIP is an important regulator of adipocyte function and changes in GIP function may contribute to progression of obesity in man [9].

In serum, both incretins, GIP and tGLP-1, are degraded by dipeptidyl peptidase IV (DPIV). The resulting short biological half-life (˜2 min in vivo) limits the therapeutic use of GIP and tGLP-1 [10,11,12]. In the case of tGLP-1, several studies have been directed at obtaining biologically active tGLP-1, analogues with improved DPIV-resistance [13,14]. For GIP, a preliminary study was performed to obtain analogues with improved DP IV-resistance [20]. Recently it was demonstrated that the full-length GIP (1-30) analogs: Tyr¹-glucitol-GIP [15] and (Pro³)GIP [20, 21] display DP IV-resistance and enhanced bioactivity.

The GIP-receptor, a member of the G-protein-coupled receptor family [16,17], has a high specificity for GIP and does not bind other peptides of the glucagon family. For this reason, GLP-1/GIP chimeric peptides show nearly no affinity for the GIP-receptor [18]. From such studies it has been concluded that the GIP₁₋₃₀ sequence of the GIP₁₋₄₂ molecule is crucial for receptor recognition. This was confirmed by Gelling et al [19] who showed that GIP₆₋₃₀-amide (GIP₆₋₃₀ₐ) contains the high affinity binding region of GIP₁₋₄₂ but exhibits antagonist activity, as do other N-terminally truncated forms.

The following patent applications have been filed related to the effects of GIP analogues on the function of various target organs and their potential use as therapeutic agents:

DE 199 21 537 discloses a method for extending the survival of insulin producing β-cells by stimulation of their proliferation and prevention of their programmed cell death. The specific goal is to increase the endogenous insulin content and insulin response to elevated blood glucose levels. An important component of this invention is the activation of protein kinase B/Akt in insulin producing β-cells in response to the administration of effectors such as GLP-1, GIP, Exendin-4 or GLP-1 receptor agonists or GIP-receptor agonists.

EP 0479 210 discloses a novel GIP analogue of the formula GIP(1-13)-X-GIP(15-30)-Y, wherein X is an amino acid residue other than Met, and Y is selected from homoserine (inclusive homoserine-lactone) and shall be referred to as “Hse”, homoserine amide (Hse-NH₂), H-Gly-Lys-Lys-Asn-Asp-Trp-Lys-His-Asn-Ile-Thr-Gln-Hse or H-Gly-Lys-Lys-Asn-Asp-Trp-Lys-His-Asn-Ile-Thr-Gln-Hse-NH₂.

WO 98/24464 discloses an antagonist of glucose-dependent insulinotropic polypeptide (GIP) consisting essentially of a 24 amino acid polypeptide corresponding to positions 7-30 of the sequence of GIP, a method of treating non-insulin dependent diabetes mellitus and a method of improving glucose tolerance in a non-insulin dependent diabetes mellitus patient.

WO 00/58360 discloses peptides, which stimulate the release of insulin. This invention especially provides a process of N terminally-modifying GIP and the use of the peptide analogues for treatment of diabetes. The specific peptide analog, which is disclosed in this invention, comprises at least 15 amino acid residues from the N terminal end of GIP (1-42). In another embodiment, Tyr¹ glucitol GIP (1-42) is disclosed.

WO 00/20592 discloses GIP or anti-idiotypic antibodies of GIP or fragments thereof as GIP-analogs for maintaining or increasing bone density or bone formation.

### REFERENCES

1. Brown, J. C., Mutt, V. and Pederson, R. A. (1970). Further purification of a polypeptide demonstrating enterogastrone activity. J Physiol (Lond) 209 (1): 57-64
2. Creutzfeidt, W. (1979) The incretin concept today. Diabetologia; 16, 75-85
3. Fehmann, H. C., Goke, B., Goke, R., et al. (1989) Synergistic stimulatory effect of glucagon-like peptide-1 (7-36) amide and glucose-dependent insulin-releasing polypeptide on the endocrine rat pancreas. FEBS Lett; 252, 109-112
4. Nauck, M. A., Niedereichhoiz, O., Ettler, R., et al. (1997) Glucagon-like peptide 1 inhibition of gastric emptying outweighs its insulinotropic effects in healthy humans. Am J Physiol; 273, E981-E988
5. Gutniak, M. K., Linde, B., Hoist, J. J., et al. (1994) Subcutaneous injection of the incretin hormone glucagon-like peptide 1 abolishes postprandial glycemia in NIDDM. Diabetes Care; 17, 1039-1044
6. Gutniak, M., Orskov, C., Hoist, J. J., et al. (1992) Antidiabetogenic effect of glucagon-like peptide-1 (7-36)amide in normal subjects and patients with diabetes mellitus [see comments]. N Engl J Med; 326, 1316
7. Nauck, M. A., Wolischiager, D., Werner, J., et al. (1996) Effects of subcutaneous glucagon-like peptide 1 (GLP-1 [7-36 amide]) in patients with NIDDM. Diabetologia; 39, 1546
8. Nauck, M. A., Kleine, N., Orskov, C., et al. (1993) Normalization of fasting hyperglycaemia by exogenous glucagon-like peptide 1 (7-36 amide) in type 2 (non-insulin-dependent) diabetic patients. Diabetologia; 36, 741-744
9. McIntosh, C. H., Bremsak, I., Lynn, F. C., et al. (1999) Glucose-dependent insulinotropic polypeptide stimulation of lipolysis in differentiated 3T3-L1 cells: wortmannin-sensitive inhibition by insulin. Endocrinology; 140, 398
10. Mentlein, R., Gallwitz, B., Schmidt, W. E. (1993) Dipeptidyl-peptidase IV hydrolyses gastric inhibitory polypeptide, glucagon-like peptide-1 (7-36)amide, peptide histidine methionine and is responsible for their degradation in human serum. Eur. J. Biochem. 214, 829-835
11. Kieffer, T. J., McIntosh, C. H., Pederson, R. A. (1995) Degradation of glucose-dependent insulinotropic polypeptide and truncated glucagon-like peptide 1 in vitro and in vivo by dipeptidyl peptidase IV. Endocrinology 136, 3585-3596
12. Pauly, R. P., Rosche, F., Wermann, M., McIntosh, C. H. S., Pederson, R. A., and Demuth, H. U. Investigation of glucose-dependent insulinotropic polypeptide-(1-42) and glucagon-like peptide-1-(7-36) degradation in vitro by dipeptidyl peptidase IV using matrix-assisted laser desorption/ionization time of flight mass spectrometry—A novel kinetic approach. J Biol Chem 271 (38), 23222-23229. 1996.
13. Deacon, C. F., Knudsen, L. B., Madsen, K., et al. (1998) Dipeptidyl peptidase IV resistant analogues of glucagon-like peptide-1 which have extended metabolic stability and improved biological activity. Diabetologia 41, 271-278
14. Siegel, E. G., Gallwitz, B., Scharf, G., et al. (1999) Biological activity of GLP-1-analogues with N-terminal modifications. Regul Pept 79, 93-102
15. O'Harte, F. P., Mooney, M. H., Flatt, P. R. (1999) NH2-terminally modified gastric inhibitory polypeptide exhibits amino-peptidase resistance and enhanced antihyperglycemic activity. Diabetes 48, 758-765
16. Gallwitz, B., Witt, M., Folsch, U. R., et al. (1993) Binding specificity and signal transduction of receptors for glucagon-like peptide-1 (7-36)amide and gastric inhibitory polypeptide on RINm5F insulinoma cells. J Mol Endocrinol 10, 259-268
17. Amiranoff, B., Vauclin-Jacques, N., Laburthe, M. (1984) Functional GIP-receptors in a hamster pancreatic beta cell line, In 111: specific binding and biological effects. Biochem Biophys Res Commun 123, 671-676
18. Gallwitz, B., Witt, M., Morys-Wortmann, C., et al. (1996) GLP-1/GIP chimeric peptides define the structural requirements for specific ligand-receptor interaction of GLP-1. Regul Pept 63, 17-22
19. Gelling, R. W., Coy, D. H., Pederson, R. A., et al. (1997) GIP(6-30amide) contains the high affinity binding region of GIP and is a potent inhibitor of GIP1-42 action in vitro. Regul Pept 69, 151-154
20. Kühn-Wache, K., Manhart, S., Hoffmann, T., et al. (2000) Analogs of Glucose-dependent insulinotropic polypeptide with increased dipeptidyl peptidase IV resistance. IN: Langner & Ansorge, Cellular peptidases in Immune Functions and Diseases 2. Kluwer Academic/Plenum Publishers, 187-195
21. Gault, V. A., O'Harte, F. P. M., Harriott, P. et al. (2002) Characterization of the cellular and metabolic effects of a novel enzyme-resistant antagonist of Glucose-dependent insulinotropic polypeptide. Biochemical and Biophysical Research Communications 290, 1420-1426.

### SUMMARY OF THE INVENTION

The present invention relates to novel C-terminally truncated fragments and novel N-terminally modified analogues of gastric inhibitory polypeptide as well as various GIP analogues with a reduced peptide bond or alterations of the amino acids close to the dipeptidyl peptidase IV (DPIV) specific cleavage site with the aim of improved DPIV-resistance and prolonging half-life. Further the invention relates to novel analogues with different linkers between potential receptor binding sites of GIP.

The compounds of the present invention and their pharmaceutically acceptable salts are useful in treating conditions in which GIP-receptor function may be altered, including non-insulin dependent diabetes mellitus and obesity. Two specific applications are proposed:
1. The compounds of the present invention are able to potentiate glucose-dependent proliferation of pancreatic β-cells.
2. The compounds of the present invention have anti-apoptotic effects on pancreatic β-cells.

### BRIEF DESCRIPTION OF THE DRAWINGS

FIG. 1: Cyclic AMP production by N-terminally modified GIP analogues in CHO-K1 cells stably transfected with the rat pancreatic islet GIP-receptor (wtGIPR cells). Stimulation was allowed to occur for 30 minutes at 37 C in 15 mM HEPES-buffered (pH 7.4) DMEM/F12+0.1% BSA and 0.5 mM IBMX with or without peptides at the concentrations shown. Cell contents were extracted in ice-cold 70% ethanol, dried in vacuo, and cyclic AMP measured by radioimmunoassay. Data represent the mean±SEM of at least three independent experiments. Data are normalized to the maximal cAMP stimulated by GIP_{1-30NH2}.

FIG. 2: Cyclic AMP production in wtGIPR cells by modified GIP1-14OH peptides, relative to native hormone. Stimulation was allowed to occur for 30 minutes at 37 C in 15 mM HEPES-buffered (pH 7.4) DMEM/F12+0.1% BSA and 0.5 mM IBMX, with or without peptides at the concentrations shown. Cell contents were extracted in ice-cold 70% ethanol, dried in vacuo, and cyclic AMP measured by radioimmunoassay. Data represent the mean±SEM of at least three independent experiments. Data are normalized to cell number.

FIG. 3: Cyclic AMP production by modified GIP1-14OH peptides (20 micrormolar) in wtGIPR cells. Data are from FIG. 2, represented as a factor of, the basal cyclic AMP content in the cells. *=P<0.05 versus 1 nM stimulated cAMP by GIP1-42; #=P<0.05 versus basal cyclic AMP (n≧3).

FIG. 4: Cyclic AMP production by GIP1-14OH peptides (40 micromolar) modified by alanine scanning. At positions 2 and 13, where alanines reside in the native primary sequence, the amino acids in those positions were replaced with those found in the primary sequence of the related hormone, glucagon. Stimulation was allowed to occur for 30 minutes at 37 C in 15 mM HEPES-buffered (pH 7.4) DMEM/F12+0.1% BSA and 0.5 mM IBMX, with or without peptides at the concentrations shown. Cell contents were extracted in ice-cold 70% ethanol, dried in vacuo, and cyclic AMP measured by radioimmunoassay. Data are represented as a factor of the basal cyclic AMP content in the cells. *=P<0.05 versus 1 nM stimulated cAMP by GIP1-42; #=P<0.05 versus basal cyclic AMP (n≧3).

FIG. 5: Cyclic AMP production wtGIPR cells by modified GIP peptides having core sequence deletions or alpha-helical insertions, relative to native hormone. Stimulation was allowed to occur for 30 minutes at 37 C in 15 mM HEPES-buffered (pH 7.4) DMEM/F12+0.1% BSA and 0.5 mM IBMX, with or without peptides at the concentrations shown. Cell contents were extracted in ice-cold 70% ethanol, dried in vacuo, and cyclic AMP measured by radioimmunoassay. Data represent the mean±SEM of at least three independent experiments. Data are normalized to cell number.

FIG. 6: Cyclic AMP production in wtGIPR cells by modified GIP peptides having core sequence deletions or alpha-helical insertions, relative to native hormone. Stimulation was allowed to occur for 30 minutes at 37 C in 15 mM HEPES-buffered (pH 7.4) DMEM/F12+0.1% BSA and 0.5 mM IBMX, with or without peptides at the concentrations shown. Cell contents were extracted in ice-cold 70% ethanol, dried in vacuo, and cyclic AMP measured by radioimmunoassay. Data represent the mean±SEM of at least three independent experiments. Data are normalized to cell number.

FIG. 7: Cyclic AMP production in wtGIPR cells by modified GIP peptides having N-terminal modifications or cyclicized between amino acids 16 and 21, relative to native hormone. Stimulation was allowed to occur for 30 minutes at 37 C in 15 mM HEPES-buffered (pH 7.4) DMEM/F12+0.1% BSA and 0.5 mM IBMX, with or without peptides at the concentrations shown. Cell contents were extracted in ice-cold 70% ethanol, dried in vacuo, and cyclic AMP measured by radioimmunoassay. Data represent the mean±SEM of at least three independent experiments. Data are normalized to the maximal cAMP produced by GIP1-42OH.

FIG. 8: Competitive binding inhibition studies on intact wtGIPR cells using ¹²⁵I-GIP versus modified GIP1-14 peptides at the concentrations shown. Equilibrium binding was achieved following 12-16 hour incubation at 4 C in 15 mM HEPES-buffered (pH 7.4) DMEM/F12+0.1% BSA+1% Trasylol (aprotinin). Unbound label was removed during washing steps, and cells were solubilized in 0.2 M NaOH and transferred to borosilicate tubes for counting cell associated radioactivity. Non-specific binding was defined as cell associated radioactivity detected in the presence of 1 micromolar GIP1-42. Data represent the mean±SEM of greater than 3 experiments, and are normalized to the specific binding of ¹²⁵I-GIP measured in the absence of competitor (Bo).

FIG. 9: Percent displacement of ¹²⁵I-GIP from wtGIPR cells by 50 micromolar peptide analogues (GIP1-14 peptides with alanine, serine, tyrosine, D-alanine, D-proline, reduced P2-P3 peptide bond, or BTD substitutions/modifications). Equilibrium binding was achieved following 12-16 hour incubation at 4 C in 15 mM HEPES-buffered (pH 7.4) DMEM/F12+0.1% BSA+1% Trasylol (aprotinin). Unbound label was removed during washing steps, and cells were solubilized in 0.2 M NaOH and transferred to borosilicate tubes for counting cell associated radioactivity. Non-specific binding was defined as cell associated radioactivity detected in the presence of 1 micromolar GIP1-42. Data represent the mean±SEM of greater than 3 experiments. *=P<0.05 versus % displacement by GIP1-14; #=P<0.05 versus zero displacement (i.e. only A3 and A5 were unable to displace measurable ¹²⁵I-GIP binding).

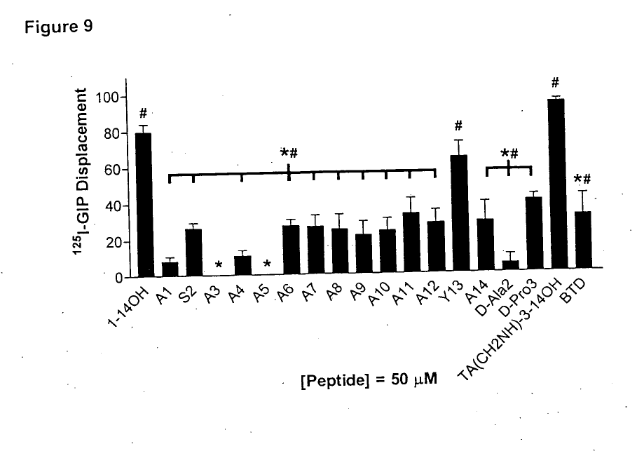

FIG. 10: Competitive binding inhibition studies on intact wtGIPR cells using ¹²⁵I-GIP versus GIP peptides having core sequence deletions or alpha-helical insertions, relative to native hormone at the concentrations shown. Equilibrium binding was achieved following 12-16 hour incubation at 4 C in 15 mM HEPES-buffered (pH 7.4) DMEM/F12+0.1% BSA+1% Trasylol (aprotinin). Unbound label was removed during washing steps, and cells were solubilized in 0.2 M NaOH and transferred to borosilicate tubes for counting cell associated radioactivity. Non-specific binding was defined as cell associated radioactivity detected in the presence of 1 micromolar GIP1-42. Data represent the mean±SEM of greater than 3 experiments, and are normalized to the specific binding of ¹²⁵I-GIP measured in the absence of competitor (Bo).

FIG. 11: Competitive binding inhibition studies on intact wtGIPR cells using ¹²⁵I-GIP versus GIP peptides having core sequence deletions or alpha-helical insertions, relative to native hormone at the concentrations shown. Equilibrium binding was achieved following 12-16 hour incubation at 4 C in 15 mM HEPES-buffered (pH 7.4) DMEM/F12+0.1% BSA+1% Trasylol (aprotinin). Unbound label was removed during washing steps, and cells were solubilized in 0.2 M NaOH and transferred to borosilicate tubes for counting cell associated radioactivity. Non-specific binding was defined as cell associated radioactivity detected in the presence of 1 micromolar GIP1-42. Data represent the mean±SEM of greater than 3 experiments, and are normalized to the specific binding of ¹²⁵I-GIP measured in the absence of competitor (Bo).

FIG. 12: Intraperitoneal glucose tolerance test in anaesthetized (65 mg/Kg sodium pentobarbital IP) male Wistar rats with synthetic GIP analogues. Intravenous (jugular) infusion of saline or peptide (A: 1 pmol/min/100 g body weight or B: 100 pmol/min/100 g body weight) was started 5 minutes prior to 1 g glucose/Kg body weight intraperitoneal injection. Blood samples were taken from the tail vein prior to infusion (basal sample) and at 10 minute intervals for one hour. Blood glucose measurements were made using hand-held glucometers. *=P<0.05 versus saline control. Data represent the mean±SEM of ≧4 animals.

FIG. 13: Oral glucose tolerance test (1 g/Kg BW) in conscious unrestrained male Wistar rats with or without subcutaneous peptide injection (8 nmol/Kg BW in 500 uL volume; or 80 nmol/Kg BW in one case). Basal samples were obtained from the tail vein prior to oral glucose and peptide injection. Samples were then obtained at the indicated time points to measure whole blood glucose using a hand held glucometer. Data represent the mean±SEM of ≧4 animals.

FIG. 14: Integrated glucose responses from conscious unrestrained male Wistar rats having concurrent oral glucose tolerance test and subcutaneous peptide injections (i.e. integrated data from FIG. 13). Area under the curve was calculated using the trapezoidal method with baseline subtraction. Data represent the mean±SEM of ≧4 animals.

FIG. 15: GIP potentiates 11 mM glucose induced cell growth to a similar level as GH (A) and GLP-1 (B) in INS-1 (832/13) cells. Cells were serum starved before and during the course of the experiment. Final cell numbers were always greater than initial plating, densities, indicative of mitogenesis, and final cell numbers were quantified fluorometrically by CYQUANT™. Values are means of 5 (A) and 4 (B) individual experiments done in triplicate, where * represents p<0.05.

FIG. 16: GIP promotes INS-1 (832/13) cell survival during glucose deprivation in a concentration-dependent manner. Cells were serum and glucose starved for 48 h, and GIP was added for the final 24 h period of culture. Final cell numbers were always less than initial plating density, indicating cell death was occurring, and final cell numbers were quantified fluorometrically by CYQUANT™. Values are means of 3 (A) and 4 (B) individual experiments done in triplicate, where * represents p<0.05.

FIG. 17: GIP promotion of INS-1 (832/13) cell survival during glucose deprivation involves p38 MAPK. Protein kinase inhibitors were added to the medium 15 min. prior to the final 24 h culture in the absence or presence of 100 nM GIP. The PKA inhibitor, H89, was unable to reverse GIP (A) or Forskolin (B) mediated cell survival. Wortmannin has deleterious effects on cell survival (C), which were partially reversed by GIP. Panel D represents the involvement of p38 MAP kinase, via specific inhibition with SB202190. Final cell numbers were quantified fluorometrically by CYQUANT™, and data represent means of 3-8 experiments done in triplicate, where * and # represent p<0.05 vs. respective controls.

FIG. 18: GIP ablates 0 mM glucose (A) and STZ (B) induced caspase-3 activity in INS-1 (832/13) cells. Cells were serum starved before and during the experiment, and 100 nM GIP, 10 □M forskolin, or 100 nM GLP-1 were added for 6 h in the presence and absence of glucose (3 mM) or STZ to assess affects on caspase-3 activity., Caspase-3 activity was quantified using the aminomethylcoumarin (AMC)-derived substrate, Z-DEVD-AMC, and correcting for total protein concentration, where * and # represent p<0.05 vs. respective controls (A, n=3; B, n=5). Relative activity was ensured to be specific by using the caspase-3 inhibitor Ac-DEVD-CHO (A, inset).

### DETAILED DESCRIPTION OF THE INVENTION

The present invention relates to novel C-terminally truncated fragments and novel N-terminally modified analogues of Glucose-dependent Insulinotropic Polypeptide as well as various GIP analogues with a reduced peptide bond or alterations of the amino acids close to the dipeptidyl peptidase IV (DPIV) specific cleavage site with the aim of improving DPIV-resistance and a prolonging half-life. The amino acid alterations according to the present invention include residues of L-amino acids, D-amino acids, proteinogenic and non-proteinogenic amino acids. Proteinogenic amino acids are defined as natural protein-derived α-amino acids. Non-proteinogenic amino acids are defined as all other amino acids, which are not building blocks of common natural proteins.

Further, the invention relates to novel analogues with different linkers between potential receptor binding sites of GIP.

More particularly, the present invention relates to novel GIP analogues with the general amino acid sequence shown in formula (1):

| | |
|---|---|
| Tyr-A-B-Gly-Thr-Phe-Ile-Ser-Asp-Tyr-Ser-Ile- | (1) |
| Ala-Met | |

wherein A and B are amino acid residues including D-amino acid residues, N-methylated amino acid residues and any other non-proteinogenic amino acid residues. Additionally, the N-terminus of the tyrosine residue in position 1 can be modified by alkylation, sulphonylation, glycation, homoserine formation, pyroglutamic acid formation, disulphide bond formation, deamidation of asparagine or glutamine residues, methylation, t-butylation, t-butyloxycarbonylation, 4-methylbenzylation, thioanysilation, thiocresylation, bencyloxymethylation, 4-nitrophenylation, bencyloxycarbonylation, 2-nitrobencoylation, 2-nitrosulphenylation, 4-toluenesulphonylation, pentafluorophenylation, diphenylmethylation, 2-chlorobenzyloxycarbonylation, 2,4,5-trichlorophenylation, 2-bromobenzyloxycarbonylation, 9-fluorenylmethyloxycarbonylation, triphenylmethylation, 2,2,5,7,8,-pentamethylchroman-6-sulphonylation, hydroxylation, oxidation of methionine, formylation, acetylation, anisylation, bencylation, bencoylation, trifluoroacetylation, carboxylation of aspartic acid or glutamic acid, phosphorylation, sulphation, cysteinylation, glycolysation with pentoses, deoxyhexoses, hexosamines, hexoses or N-acetylhexosamines, farnesylation, myristolysation, biotinylation, palmitoylation, stearoylation, geranylgeranylation, glutathionylation, 5′-adenosylation, ADP-ribosylation, modification with N-glycolylneuraminic acid, N-acetylneuraminic acid, pyridoxal phosphate, lipoic acid, 4′-phosphopantetheine, and N-hydroxysuccinimide. The peptide of formula 1 can be modified by the introduction of at least one E-amino fatty acid acylated lysine in any amino acid position.

The sequence of native GIP (1-14) is excluded from the present invention.

The most preferred compounds of formula (1) are D-Ala²-GIP (1-14), Pro³-GIP (1-14) and Ser²-GIP (1-14).

In another preferred embodiment the present invention relates to GIP analogues with a reduced peptide bond, shown by formula (2) of

| | |
|---|---|
| Tyr-Ala-Ψ(CH₂NH₂)-Glu-Gly-Thr-Phe-Ile-Ser- | (2a) |
| Asp-Tyr-Ser-Ile-Ala-Met | |
| Tyr-Ala-ψ(CH₂NH)-Glu-Gly-Thr-Phe-Ile-Ser-Asp- | (2b) |
| Tyr-Ser-Ile-Ala-Met-Asp-Lys-Ile-His-Gln-Gln- | |
| Asp-Phe-Val-Asn-Trp-Leu-Leu-Ala-Gln-Lys | |

In a further embodiment, the present invention relates to a novel GIP analogue with the general amino acid sequence shown by formula (3) of

| | |
|---|---|
| Tyr-Ala-Glu-Gly-Thr-Phe-Ile-Ser-Asp-Tyr-Ser- | (3) |
| Ile-Tyr-Met | |

In another embodiment, the present invention provides novel GIP analogues of formulas 4a-4l as result of an alanine scan. In particular, these are

| | |
|---|---|
| Ala-Ala-Glu-Gly-Thr-Phe-Ile-Ser-Asp-Tyr-Ser- | (4a) |
| Ile-Ala-Met | |
| Tyr-Ala-Ala-Gly-Thr-Phe-Ile-Ser-Asp-Tyr-Ser- | (4b) |
| Ile-Ala-Met | |
| Tyr-Ala-Glu-Ala-Thr-Phe-Ile-Ser-Asp-Tyr-Ser- | (4c) |
| Ile-Ala-Met | |
| Tyr-Ala-Glu-Gly-Ala-Phe-Ile-Ser-Asp-Tyr-Ser- | (4d) |
| Ile-Ala-Met | |
| Tyr-Ala-Glu-Gly-Thr-Ala-Ile-Ser-Asp-Tyr-Ser- | (4e) |
| Ile-Ala-Met | |
| Tyr-Ala-Glu-Gly-Thr-Phe-Ala-Ser-Asp-Tyr-Ser- | (4f) |
| Ile-Ala-Met | |
| Tyr-Ala-Glu-Gly-Thr-Phe-Ile-Ala-Asp-Tyr-Ser- | (4g) |
| Ile-Ala-Met | |
| Tyr-Ala-Glu-Gly-Thr-Phe-Ile-Ser-Ala-Tyr-Ser- | (4h) |
| Ile-Ala-Met | |

Novel GIP analogues can be obtained by synthesis of linker peptides. Therefore, the present invention provides linker peptides according to formula (5):

| | |
|---|---|
| Tyr-A-B-Gly-Thr-Phe-C-Gln-Gln-Asp-Phe-Val-Asn- | (5) |
| Trp-Leu-Leu-Ala-Gln-Lys-Gly-Lys-Lys-Asn-Asp- | |
| Trp-Lys-His-Asn-Ile-Thr-Gln | |

wherein C is
a) not used,
b) a linker peptide consisting of 4 amino acid residues. Any combination of amino acid residues, including residues of D-amino acids and non-proteinogenic amino acids, is allowed and within the scope of the present invention,
c) Glu-Lys-Glu-Lys,
d) Ala-Ala-Ala-Ala,
e) a linker peptide consisting of 12 amino acid residues. Any combination of amino acid residues, including residues of D-amino acids and non-proteinogenic amino acids, is allowed and within the scope of the present invention,
f) Glu-Lys-Glu-Glu-Lys-Glu-Lys-Glu-Glu-Lys-Glu-Lys,
e) 6-Ahxₙ (6-aminohexanoic acid) with n=1-3, or
f) Omega-amino fatty acids (saturated and unsaturated) ω-NH2—(CHx)n-COOH with n=6-21;
   and wherein A and B are amino acid residues including D-amino acid residues, N-methylated amino acid residues and any other non-proteinogenic amino acid residues.

The N-terminus of the tyrosine residue in position 1 can be modified by alkylation, sulphonylation, glycation, homoserine formation, pyroglutamic acid formation, disulphide bond formation, deamidation of asparagine or glutamine residues, methylation, t-butylation, t-butyloxycarbonylation, 4-methylbenzylation, thioanysilation, thiocresylation, bencyloxymethylation, 4-nitrophenylation, bencyloxycarbonylation, 2-nitrobencoylation, 2-nitrosulphenylation, 4-toluenesulphonylation, pentafluorophenylation, diphenylmethylation, 2-chlorobenzyloxycarbonylation, 2,4,5-trichlorophenylation, 2-bromobenzyloxycarbonylation, 9-fluorenylmethyloxycarbonylation, triphenylmethylation, 2,2,5,7,8,-pentamethylchroman-6-sulphonylation, hydroxylation, oxidation of methionine, formylation, acetylation, anisylation, bencylation, bencoylation, trifluoroacetylation, carboxylation of aspartic acid or glutamic acid, phosphorylation, sulphation, cysteinylation, glycolysation with pentoses, deoxyhexoses, hexosamines, hexoses or N-acetylhexosamines, farnesylation, myristolysation, biotinylation, palmitoylation, stearoylation, geranylgeranylation, glutathionylation, 5′-adenosylation, ADP-ribosylation, modification with N-glycolylneuraminic acid, N-acetylneuraminic acid, pyridoxal phosphate, lipoic acid, 4′-phosphopantetheine, and N-hydroxysuccinimide. Further, the introduction of a reduced peptide bond or any other modification of the peptide bond between position 2 and 3 is provided. The peptide of formula 5 can be modified by the introduction of at least one ε-amino fatty acid acylated lysine in any amino acid position.

Further, the present invention provides linker peptides according to formula (6):

| | |
|---|---|
| Tyr-A-B-Gly-Thr-Phe-Ile-Ser-Asp-Tyr-Ser-Ile- | (6) |
| Ala-Met-D-Gln-Gln-Asp-Phe-Val-Asn-Trp-Leu-Leu- | |
| Ala-Gln-Lys-Gly-Lys-Lys-Asn-Asp-Trp-Lys-His- | |
| Asn-Ile-Thr-Gln | |

wherein D is
g) unused,
h) a linker peptide consisting of 4 amino acid residues. Any combination of amino acid residues, including residues of D-amino acids and non-proteinogenic amino acids, is possible and within the scope of the present invention, is allowed and within the scope of the present invention.
i) Ala-Ala-Ala-Ala,
j) Glu-Lys-Glu-Lys
k) 6-Ahxₙ (6-aminohexanoic acid) with n=1-3, or
l) Omega-amino fatty acids (saturated and unsaturated) ω-NH2—(CHx)n-COOH with n=6-21; and
   wherein A and B are amino acid residues including D-amino acid residues, N-methylated amino acid residues and any other non-proteinogenic amino acid residues.

The N-terminus of the tyrosine residue in position 1 can be modified by alkylation, acetylation and glycation. Further, the introduction of a reduced peptide bond or any other modification of the peptide bond between position 2 and 3 is provided. The peptide of formula 6 can be modified by the introduction of at least one ε-amino fatty is acid acylated lysine in any amino acid position.

Other novel GIP analogues can be obtained by phosphorylation of Ser². Preferred compounds of the present invention are those of formulas 7a-7c:

Novel GIP analogues of formulas 7a-7c, comprising a phosphorylated seryl residue:

| | |
|---|---|
| Tyr-[Ser(P)]-Glu-Gly-Thr-Phe-Ile-Ser-Asp-Tyr- | (7a) |
| Ser-Ile-Ala-Met | |
| Tyr-[Ser(P)]-Glu-Gly-Thr-Phe-Ile-Ser-Asp-Tyr- | (7b) |
| Ser-Ile-Ala-Met-Asp-Lys-Ile-His-Gln-Gln-Asp- | |
| Phe-Val-Asn-Trp-Leu-Leu-Ala-Gln-Lys | |

Further, novel GIP analogues are constrained GIP analogues by introduction of side-chain lactam bridges between Asp/Glu- and Lys-residues of the peptide sequence. One preferred compound of the present invention is [Cyclo(Lys¹⁶, Asp²¹)] GIP (1-30) as of formula 8

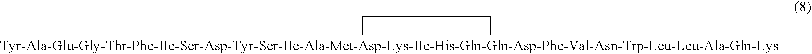

The present invention further includes within its scope both the amide and the free carboxylic acid forms of the compounds of this invention. In view of the close relationship between the free compounds and the compounds in the form of their amides, whenever a compound is referred to in this context, the amide as well as the free carboxylic acid form is intended, provided such is possible or appropriate under the circumstances.

The compounds of the present invention can be converted into acid addition salts, especially pharmaceutically acceptable acid addition salts. The pharmaceutically acceptable salt generally takes a form in which an amino acids basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. All pharmaceutically acceptable acid addition salt forms of the compounds of the present invention are intended to be embraced by the scope of this invention.

In view of the close relationship between the free compounds and the compounds in the form of their salts, whenever a compound is referred to in this context, a corresponding salt is also intended, provided such is possible or appropriate under the circumstances.

The present invention further includes within its scope prodrugs of the compounds of this invention. In general, such prodrugs will be functional derivatives of the compounds which are readily convertible in vivo into the desired therapeutically active compound. Thus, in these cases, the methods of treatment of the present invention, the term “administering” shall encompass the treatment of the various disorders described with prodrug versions of one or more of the claimed compounds, but which converts to the above specified compound in vivo after administration to the subject. Conventional procedures for the selection and preparation of suitable prodrug derivatives are described, for example, in “Design of Prodrugs”, ed. H. Bundgaard, Elsevier, 1985 and the patent applications DE 198 28 113 and DE 198 28 114, which are fully incorporated herein by reference.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms of the compounds may exist as polymorphs and as such are intended to be included in the present invention. In addition, some of the compounds may form solvates with water (i.e. hydrates) or common organic solvents, and such solvates are also intended to be encompassed within the scope of this invention.

The compounds, including their salts, can also be obtained in the form of their hydrates, or include other solvents used for their crystallization.

Several compounds of the present invention, including their corresponding pharmaceutically acceptable salts, are characterized in that they have an improved resistance against degradation by the enzyme activity of dipeptidyl peptidase IV (DP IV) or DP IV-like enzymes. DP IV is present in a wide variety of mammalian organs and tissues e.g. the intestinal brush-border (Gutschmidt S. et al., “In situ”—measurements of protein contents in the brush border region along rat jejunal villi and their correlations with four enzyme activities. Histochemistry 1981, 72 (3), 467-79), exocrine epithelia, hepatocytes, renal tubuli, endothelia, myofibroblasts (Feller A. C. et al., A monoclonal antibody detecting dipeptidyl peptidase IV in human tissue. Virchows Arch. A. Pathol. Anat. Histopathol. 1986; 409 (2): 263-73), nerve cells, lateral membranes of certain surface epithelia, e.g. Fallopian tube, uterus and vesicular gland, in the luminal cytoplasm of e.g., vesicular gland epithelium, and in mucous cells of Brunner's gland (Hartel S. et al., Dipeptidyl peptidase (DPP) IV in rat organs. Comparison of immunohistochemistry and activity histochemistry. Histochemistry 1988; 89 (2): 151-61), reproductive organs, e.g. cauda epididymis and ampulla, seminal vesicles and their secretions (Agrawal & Vanha-Perttula, Dipeptidyl peptidases in bovine reproductive organs and secretions. Int. J. Androl. 1986, 9 (6): 435-52). In human serum, two molecular forms of dipeptidyl peptidase are present (Krepela E. et al., Demonstration of two molecular forms of dipeptidyl peptidase IV in normal human serum. Physiol. Bohemoslov. 1983, 32 (6): 486-96). The serum high molecular weight form of DP IV is expressed on the surface of activated T cells (Duke-Cohan J. S. et al., Serum high molecular weight dipeptidyl peptidase IV (CD26) is similar to a novel antigen DPPT-L released from activated T cells. J. Immunol. 1996, 156 (5): 1714-21). In one embodiment of the present invention, all molecular forms, homologues and epitopes of DP IV from all mammalian tissues and organs, also of those, which are undiscovered yet, are intended to be embraced by the scope of this invention.

Among the rare group of proline-specific proteases, DP IV was originally believed to be the only membrane-bound enzyme specific for proline as the penultimate residue at the amino-terminus of the polypeptide chain. However, other molecules have been identified recently that are structurally non-homologous with DP IV, but exhibit corresponding enzyme activity. Among the DP IV-like enzymes identified so far are fibroblast activation protein α, dipeptidyl peptidase IV β, dipeptidyl aminopeptidase-like protein, N-acetylated α-linked acidic dipeptidase, quiescent cell proline dipeptidase, dipeptidyl peptidase II, attractin and dipeptidyl peptidase IV related protein (DPP 8), and these are described in the review article by Sedo & Malik (Sedo & Malik, Dipeptidyl peptidase IV-like molecules: homologous proteins or homologous activities? Biochimica et Biophysica Acta 2001, 36506: 1-10). In another preferred embodiment of the present invention, all molecular forms, homologues and epitopes of proteins comprising DP IV-like enzyme activity, from all mammalian tissues and organs, also of those, which are undiscovered yet, are intended to be embraced by the scope of this invention.

The common property of the compounds of the present invention, including their corresponding pharmaceutically acceptable salts, is their improved resistance against degradation by the enzyme activity of DP IV or DP IV like enzymes that can be measured by MALDI-TOF mass spectrometry. The results for selected GIP analogues according to the present invention are shown in table 1 to example 3. It was demonstrated by MALDI-TOF-MS that the substitution of amino acids in the cleavage position by D-Ala², NMeGlu³, Pro³ or the introduction of a reduced peptide leads to resistance against DPIV degradation for up to 24 hours in GIP₁₋₃₀ analogs as well as in the corresponding GIP₁₋₁₄ analogs. Analogs with Val-, Gly-, Ser-substitution for Ala² or D-Glu-substitution for Glu³ showed reduced hydrolysis rates by DPIV. For the results see also table 1.

TABLE 1
N-terminal sequences, masses and DPIV-resistance of synthetic GIP analogs

| | | | | half life after |
|---|---|---|---|---|
| | | Mass (M) | MALDI | incubation with |
| GIP-analog | N-terminal sequence | calculated | M + H⁺ | DP IV |
| GIP₁₋₄₂ₐ | Tyr-Ala-Glu-Gly . . . | 4983.64 | 4983.9 | Not determined |
| GIP₁₋₃₀ₐ | Tyr-Ala-Glu-Gly . . . | 3552.02 | 3553.3 | <15 min^{a} |
| GIP₃₋₄₂ₐ | Glu-Gly . . . | 4749.38 | 4751.4 | Not determined |
| D-Ala²-GIP₁₋₃₀ₐ | Tyr-D-Ala-Glu-Gly . . . | 3552.02 | 3553.8 | stable |
| N-MeGlu³-GIP₁₋₃₀ₐ | Tyr-Ala-MeGlu-Gly . . . | 3565.07 | 3566.1 | stable |
| D-Glu³-GIP₁₋₃₀ | Tyr-Ala-D-Glu-Gly . . . | 3551.07 | 3553.0 | 40.3 ± 4.8 |
| Pro³-GIP₁₋₃₀ | Tyr-Ala-Pro-Gly . . . | 3519.07 | 3522.9 | stable |
| Ser²-GIP₁₋₃₀ₐ | Tyr-Ser-Glu-Gly . . . | 3567.07 | 3568.0 | 137.1 ± 12.3 |
| Val²-GIP₁₋₃₀ₐ | Tyr-Val-Glu-Gly . . . | 3579.12 | 3580.7 | 298.3 ± 92.2 |
| Gly²-GIP₁₋₃₀ₐ | Tyr-Gly-Glu-Gly . . . | 3537.04 | 3539.1 | 150.5 ± 27.3 |
| YAψ(CH2NH)-GIP₃₋₃₀ₐ | Tyr-Alaψ(CH2NH)- | 3537.07 | 3539.0 | stable |
| | Glu-Gly . . . | | | |
| GIP₁₋₆ₐ | Tyr-Ala-Glu-Gly . . . | 685.74 | 686.9 | >7.5 min |
| D-Ala²-GIP₁₋₆ₐ | Tyr-D-Ala-Glu-Gly . . . | 685.74 | 686.7 | stable |
| Gly²-GIP₁₋₆ₐ | Tyr-Gly-Glu-Gly . . . | 671.71 | 672.0 | Not detectable^{b} |
| Ser²-GIP₁₋₆ₐ | Tyr-Ser-Glu-Gly . . . | 701.74 | 702.0 | 79.0 ± 12.2 |
| Pro²-GIP₁₋₆ₐ | Tyr-Pro-Glu-Gly . . . | 711.78 | 712.7 | >7.5 min |
| Val²-GIP₁₋₆ₐ | Tyr-Val-Glu-Gly . . . | 713.79 | 715.2 | Not detectable |
| Pro₃-GIP₁₋₆ₐ | Tyr-Ala-Pro-Gly . . . | 653.78 | 655.0 | stable |
| YAψ(CH2NH)-GIP₃₋₁₄ₐ | Tyr-Alaψ(CH2NH)- | 1553.75 | 1555.7 | stable |
| | Glu-Gly . . . | | | |
| Pro³-GIP₁₋₁₄ | Tyr-Ala-Pro-Gly . . . | 1535.75 | 1534.0 | stable |
| D-Ala²-GIP₁₋₁₄ | Tyr-D-Ala-Glu-Gly . . . | 1567.75 | 1570.6 | stable |
| GIP₁₋₁₃ | Tyr-Ala-Glu-Gly . . . | 1435.57 | 1435.6 | 11.5 ± 2.5 |
| GIP₁₋₁₅ | Tyr-Ala-Glu-Gly . . . | 1681.85 | 1682.6 | 35.0 ± 5.2 |
| GIP₁₅₋₃₀ₐ | Asp-Lys-Ile-Arg . . . | 2001.34 | 2003.3 | Not determined |
| GIP₁₇₋₃₀ₐ | Ile-Arg-Gln-Gln | 1758.07 | 1761.1 | Not determined |
| GIP₁₉₋₃₀ₐ | Gln-Gln-Asp-Phe | 1488.72 | 1489.8 | Not determined |
| GIP₇₋₃₀ₐ | Ile-Ser-Asp-Tyr | 2882.31 | 2886.9 | 130.1 ± 10.6 |

^{a}After 15 min are 92% of GIP₁₋₃₀ hydrolyzed
^{b}After 1500 min only 25% of G²GIP₁₋₃₀ are degraded

In another preferred embodiment, the compounds of the present invention, including their corresponding pharmaceutically acceptable salts, are characterized by their ability to bind to the GIP-receptor. The ability of the compounds of the present invention, including their corresponding pharmaceutically acceptable salts to bind to the GIP-receptor can be measured employing binding studies using ¹²⁵I-labeled spGIP₁₋₄₂ such as pursuant to the method described in example 4.

The displacement studies do not show non-specific binding of the compounds to the receptor. This is a term used to describe binding remaining in the presence of excess (≧1 μM) GIP₁₋₄₂ (or GIP₁₋₃₀). This value has already been subtracted from data presented.

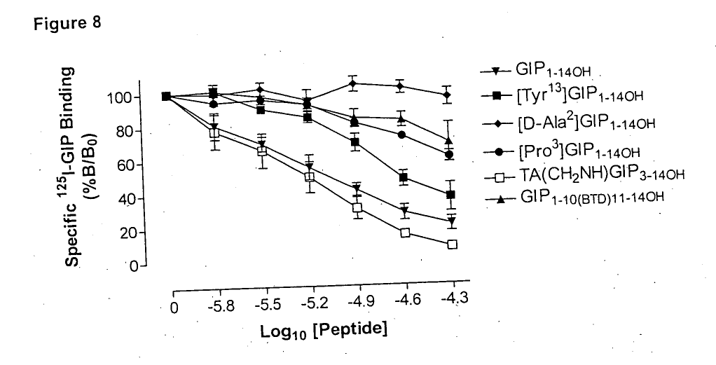

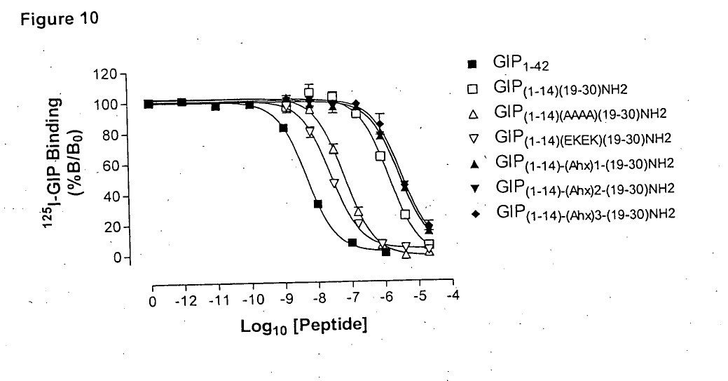

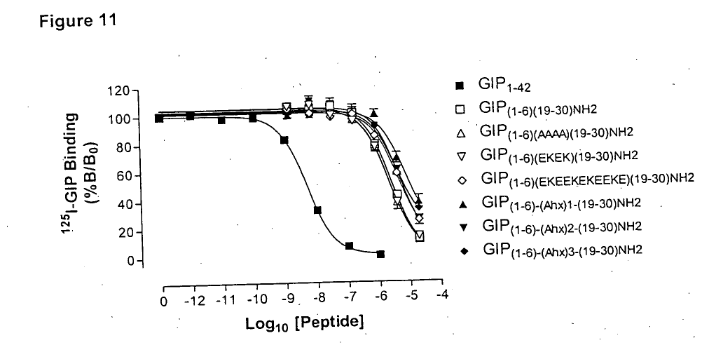

Examples of compounds of the present invention that bind and displace ¹²⁵I-GIP₁₋₄₂ from the GIP-receptor are shown in FIGS. 8, 10 and 11 and in Tables 2 and 3.

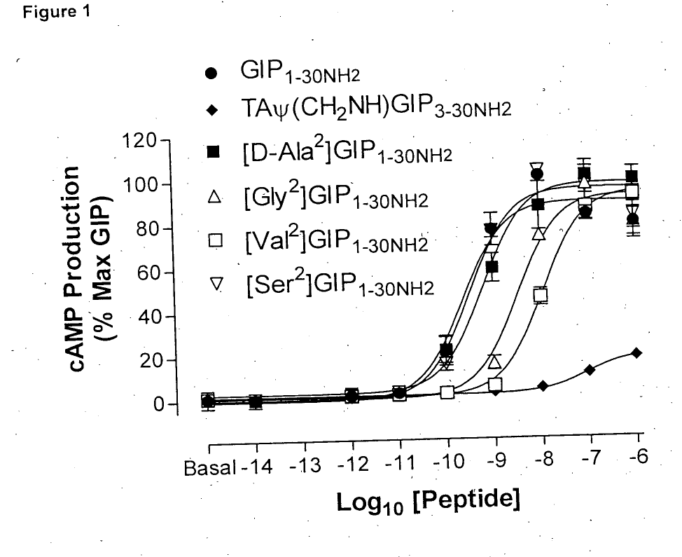

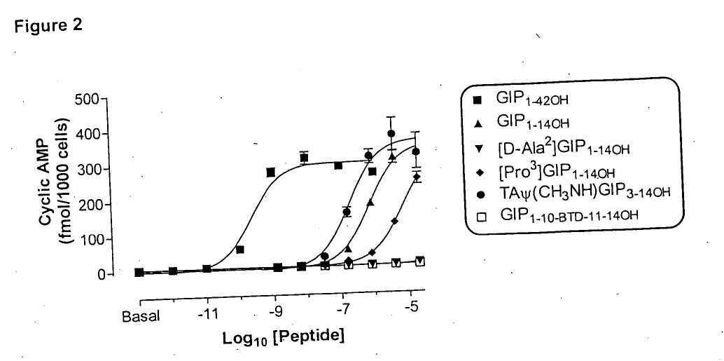

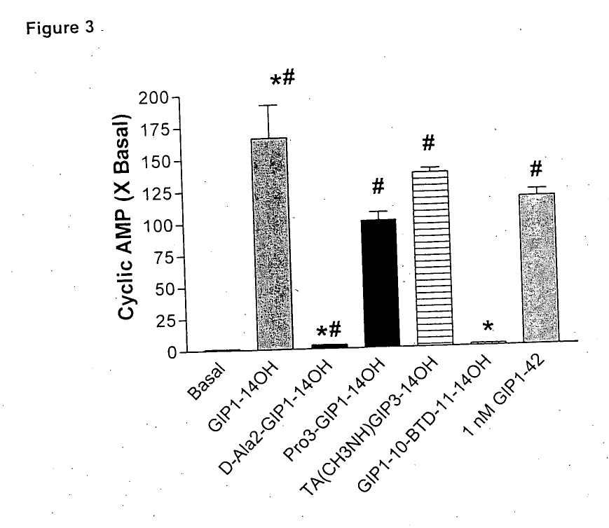

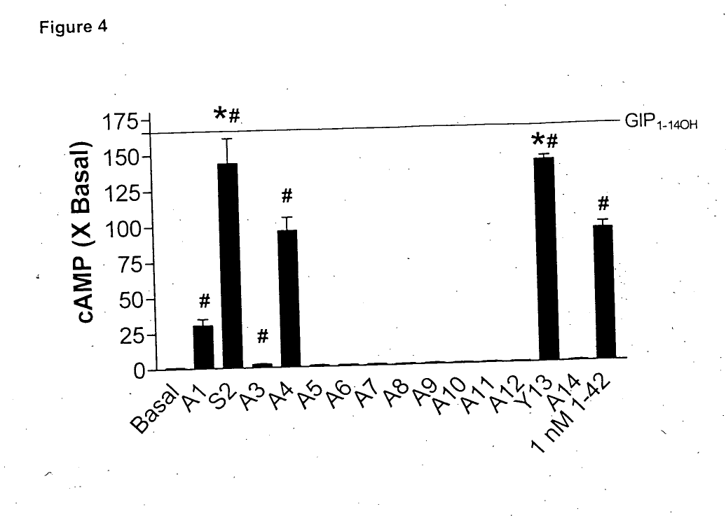

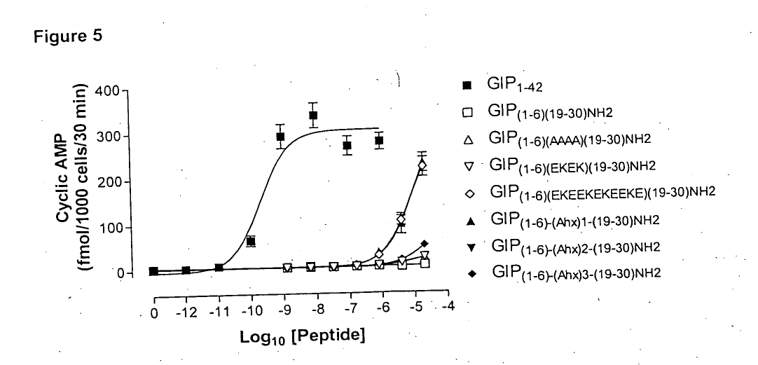

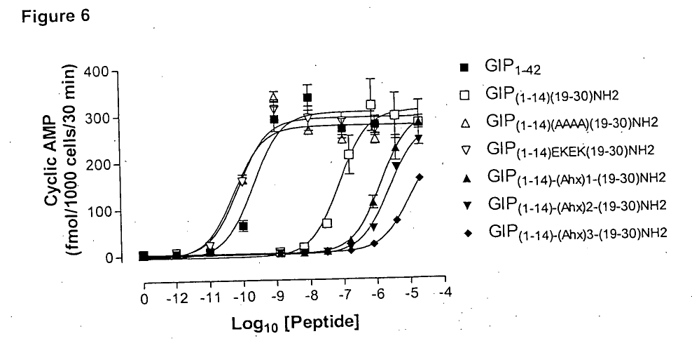

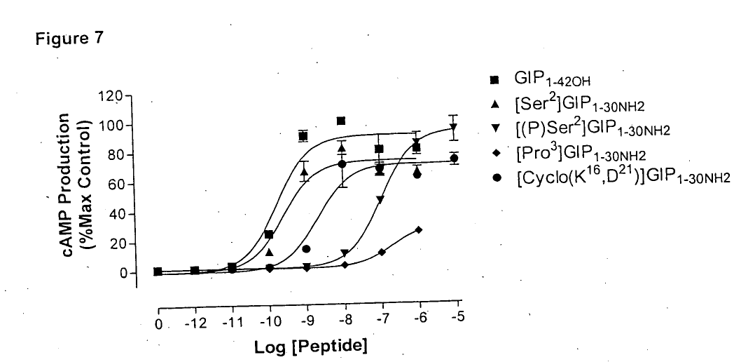

Surprisingly, the compounds of the present invention are functionally active. The biological activity of the compounds of the present invention, including their corresponding pharmaceutically acceptable salts, can be measured by determining the production of cyclic AMP following receptor binding. The cAMP production assay is described in example 4. Substitution of D-Glu for Glu³ and D-Ala for Ala² resulted in peptides with only small reductions in their ability to stimulate adenylyl cyclase whereas the Val²- and Gly²-analogs showed a significant reduction in efficacy. Interestingly, the introduction, of the reduced peptide bond resulted in a dramatic deterioration of cAMP production. This confirms the importance of the integrity of the N-terminus of GIP. Further results are shown in Tables 2 and 3- and in FIGS. 1-7.

TABLE 2
Cyclic AMP production and competitive binding displacement studies on
GIP analogs of variable length

| | | | Receptor Binding | |
|---|---|---|---|---|
| | cAMP Production | | % Displace- | |
| Synthetic | (Fold Basal^{a}) | | ment at | |
| Peptide: | 10 μM | 20 μM | 10 μM | IC₅₀ (nM) |
| GIP(1-42)_{OH} | 119 ± 11 | — | 100 | 3.2 ± 0.3 |
| 1-6_{NH2} | 1.27 ± 0.18 | 1.08 ± 0.03 | −3.6 ± 7.8 | — |
| 1-7_{NH2} | 0.92 ± 0.05 | 1.06 ± 0.06 | −6.1 ± 3.4 | — |
| 1-13_{OH} | 1.03 ± 0.06 | 1.15 ± 0.07 | −0.2 ± 3.4 | — |
| 1-13_{NH2} | 6.51 ± 1.33 | 15.7 ± 3.0 | 5.0 ± 1.1* | — |
| 1-14_{OH} | 88.9 ± 9.5 | 85.2 ± 7.6 | 51.3 ± 1.2 | — |
| 1-14_{NH2} | 75.4 ± 10.7 | 88.3 ± 5.9 | 27.9 ± 2.8 | — |
| 1-15_{OH} | 0.97 ± 0.06 | 0.91 ± 0.05 | −3.1 ± 4.3 | — |
| 1-15_{NH2} | 2.26 ± 0.32 | 4.37 ± 0.51* | 4.2 ± 1.7 | — |
| 1-30_{NH2} | 108 ± 12 | —^{c} | 99.8 ± 1.2 | 2.0 ± 0.7 |
| 7-30_{NH2} | 0.89 ± 0.06 | 0.85 ± 0.03 | 99.3 ± 1.0 | 23.7 ± 3.7 |
| 15-42_{OH} | 1.02 ± 0.10 | 1.01 ± 0.03 | 83.3 ± 0.7 | 1270 ± 150 |
| 15-30_{NH2} | 1.24 ± 0.28 | 1.01 ± 0.11 | 82.7 ± 1.0 | 1400 ± 310 |
| 16-30_{NH2} | 1.04 ± 0.06 | 0.80 ± 0.02 | 82.1 ± 1.9 | 2530 ± 450 |
| 17-30_{NH2} | 1.13 ± 0.09 | 1.12 ± 0.05 | 81.9 ± 2.1 | 1540 ± 550 |
| 19-30_{NH2} | 20.1 ± 1.3 | 45.0 ± 1.6 | 52.3 ± 0.6 | — |

*p < 0.05
^{a}Basal cyclic AMP = 2.737 ± 0.079 fmol/1000 cells;
^{b}cyclic AMP stimulated by 20 μM peptide, if plateau levels were not achieved
^{c}By definition, 10 μM GIP₁₋₄₂ displaces all specific ¹²⁵I-GIP binding.
^{d}Estimated using maximal GIP₁₋₄₂-stimulated cAMP.

TABLE 3
Summary statistics for cyclic AMP production and competitive binding
displacement studies on synthetic GIP fragments using CHO-K1 cells
transfected with the rat GIP-receptor. Data represent mean ± S.E.M. of 3
independent experiments.

| | Molecular Weight | | cAMP Production | | Receptor Binding | |
|---|---|---|---|---|---|---|
| | (Daltons) | | Max. cAMP^{a} | | % Displacement | |
| Synthetic Peptide: | Expected | Measured | (Fold Basal) | EC₅₀ | at 20 μM | IC₅₀ |
| GIP_{(1-42OH)} | 4984.3 | 4984.7 | 122 ± 10 | 231 ± 34 pM | 100^{b} | 4.18 ± 0.47 nM |
| GIP_{(1-6)(19-30)NH2} | 2157.6 | 2158.8 | 1.81 ± 0.42* | — | 88.2 ± 0.7 | 2.74 ± 0.37 □M |
| GIP_{(l-6)(AAAA)(19-30)NH2} | 2441.8 | 2440.5 | 7.21 ± 0.99* | — | 88.7 ± 3.0 | 2.41 ± 0.46 □M |
| GIP_{(1-6)(EKEK)(19-30)NH2} | 2672.1 | 2674.1 | 8.17 ± 0.87* | — | 86.8 ± 1.6 | 2.09 ± 0.23 □M |
| GIP_{(1-6)(EKEEKEKEEKE)(19-30)NH2} | 3574.0 | 3575.9 | 84.9 ± 8.1* | 8.39 ± 0.18 μM*^{d} | 75.1 ± 2.7 | 4.27 ± 0.14 □M |
| GIP_{(1-6)(Ahx)1(19-30)NH2} | 2270.7 | 2274.0 | 95.9 ± 8.6* | 14.5 ± 4.7 μM*^{d} | 62.0 ± 4.3 | 8.73 ± 2.24 □M |
| GIP_{(1-6)(Ahx)2(19-30)NH2} | 2383.8 | 2386.0 | 2.55 ± 0.84* | — | 75.3 ± 3.3 | 4.98 ± 0.40 □M |
| GIP_{(1-6)(Ahx)3(19-30)NH2} | 2497.0 | 2498.8 | 13.5 ± 1.5* | — | 67.1 ± 1.0 | 4.03 ± 0.64 □M |
| GIP_{(1-14)(19-30)NH2} | 3038.6 | 3040.6 | 127 ± 22 | 78.7 ± 2.3 nM* | 95.4 ± 0.7 | 1.37 ± 0.06 □M |
| GIP_{(1-14)(AAAA)(19-30)NH2} | 3322.9 | 3328.3 | 82.1 ± 2.8* | 58.7 ± 2.7 pM* | 100.0 ± 0.9 | 66.3 ± 7.5 nM |
| GIP_{(1-14)(EKEK)(19-30)NH2} | 3553.0 | 3551.6 | 80.6 ± 5.6* | 77.0 ± 6.1 pM* | 98.7 ± 1.1 | 26.0 ± 1.6 nM |
| GIP_{(1-14)(Ahx)1(19-30)NH2} | 3151.6 | 3155.6 | 102.1 ± 5.0 | 1.41 ± 0.32 μM*^{d} | 86.1 ± 1.9 | 2.71 ± 0.23 □M |
| GIP_{(1-14)(Ahx)2(19-30)NH2} | 3264.9 | 3264.8 | 95.9 ± 3.2* | 2.51 ± 0.25 μM*^{d} | 85.8 ± 1.6 | 2.77 ± 0.14 □M |
| GlP_{(1-14)(Ahx)3(19-30)NH2} | 3377.9 | 3389.4 | 49.5 ± 1.6* | ˜20 μM*^{d} | 82.7 ± 3.2 | 3.21 ± 0.44 □M |

*p <0.05
^{a}Basal cyclic AMP = 2.737 ± 0.079 fmol/1000 cells;
^{b}cyclic AMP stimulated by 20 μM peptide, if plateau levels were not achieved
^{c}By definition, 10 μM GIP₁₋₄₂ displaces all specific ¹²⁵I-GIP binding.
^{d}Estimated using maximal GIP₁₋₄₂-stimulated cAMP.

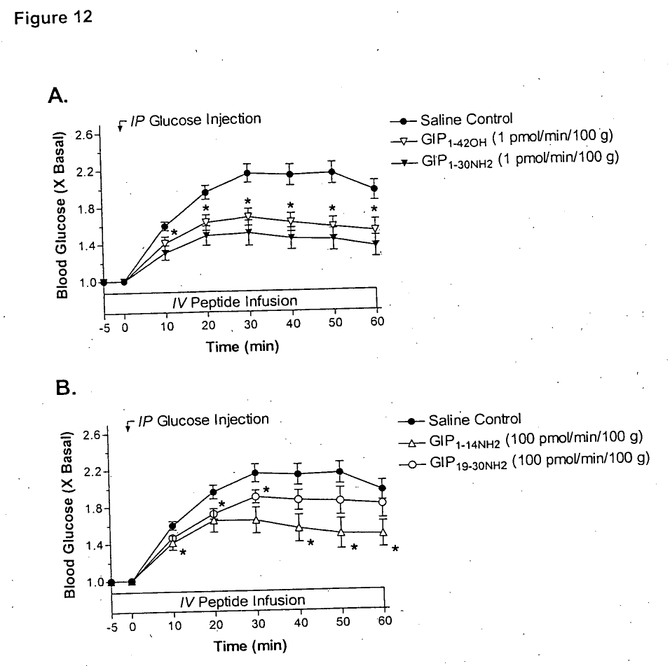

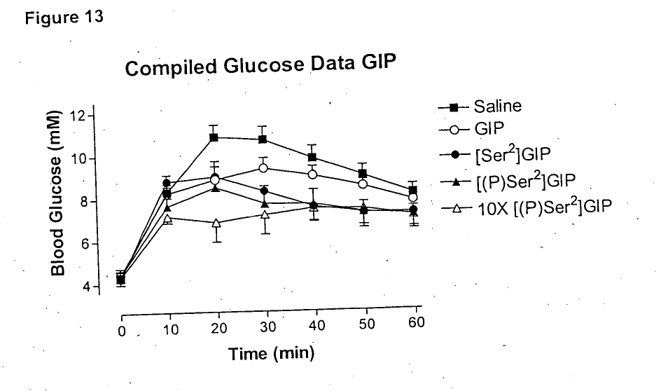

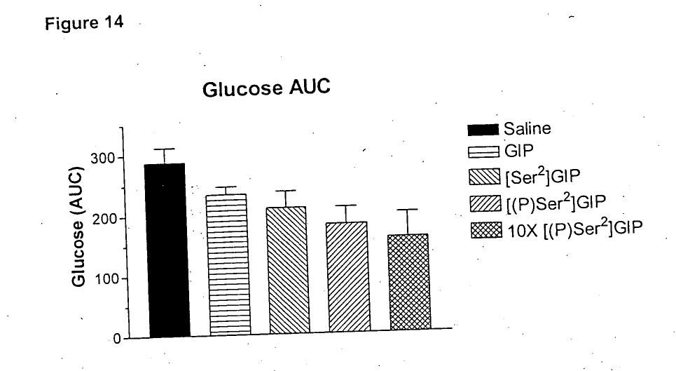

Based on their functional activity in vitro, compounds of the present invention were tested for their ability to improve glucose tolerance and decrease glucose AUC in mammals in vivo and therefore are useful for the treatment of non-insulin dependent diabetes mellitus (NIDDM). The ability of the compounds, including their corresponding pharmaceutically acceptable salts, to improve glucose tolerance in a mammal and to decrease glucose AUC can be measured employing the Wistar rat model. The method is described in Example 5. Results are shown in FIGS. 12, 13 and 14.

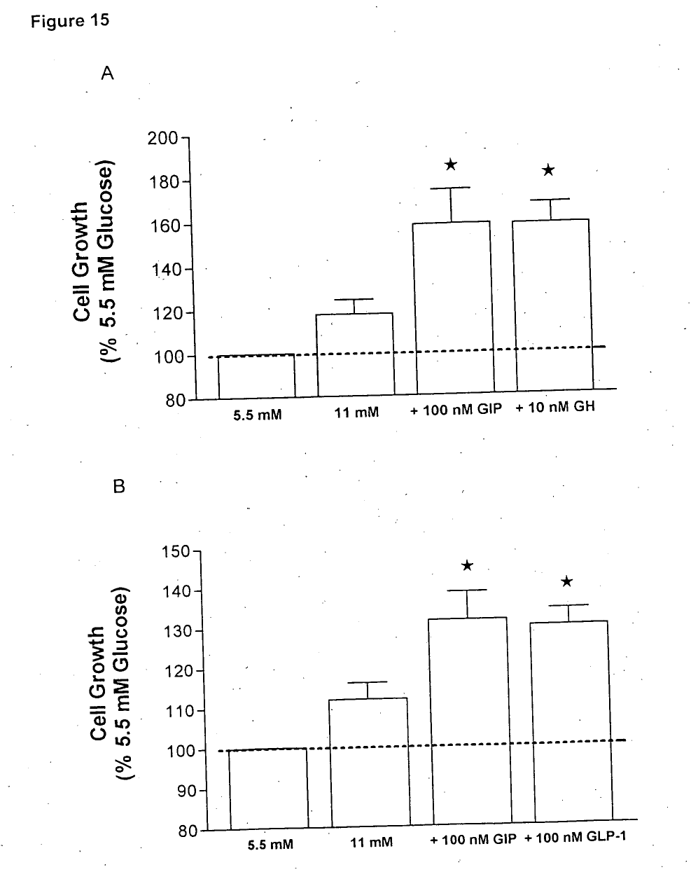

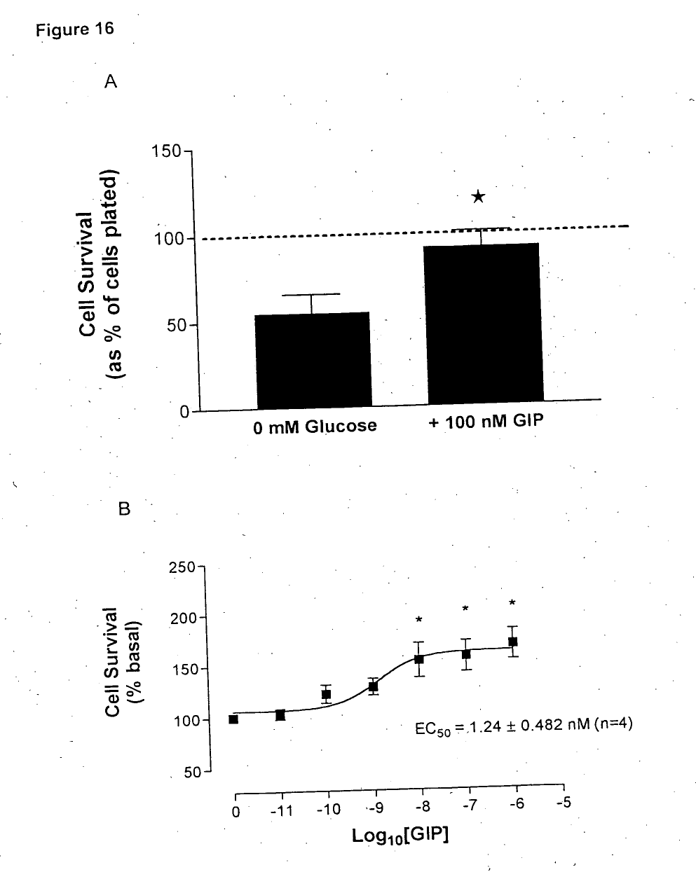

Based on their receptor binding capabilities and their stimulatory effect on cAMP release, it was found that the compounds of the present invention are able to potentiate glucose dependent proliferation of pancreatic β-cells. Surprisingly, and as an especially preferred embodiment, the compounds of the present invention show, independently from the presence of glucose, a concentration-dependent effect on the β-cell survival. The ability of the compounds of the present invention, including their corresponding pharmaceutically acceptable salts, to potentiate glucose dependent, β-cell proliferation as well as glucose independent β-cell survival can be measured employing an assay with INS-1 cells as described in Example 6. Results are shown in FIGS. 15 and 16.

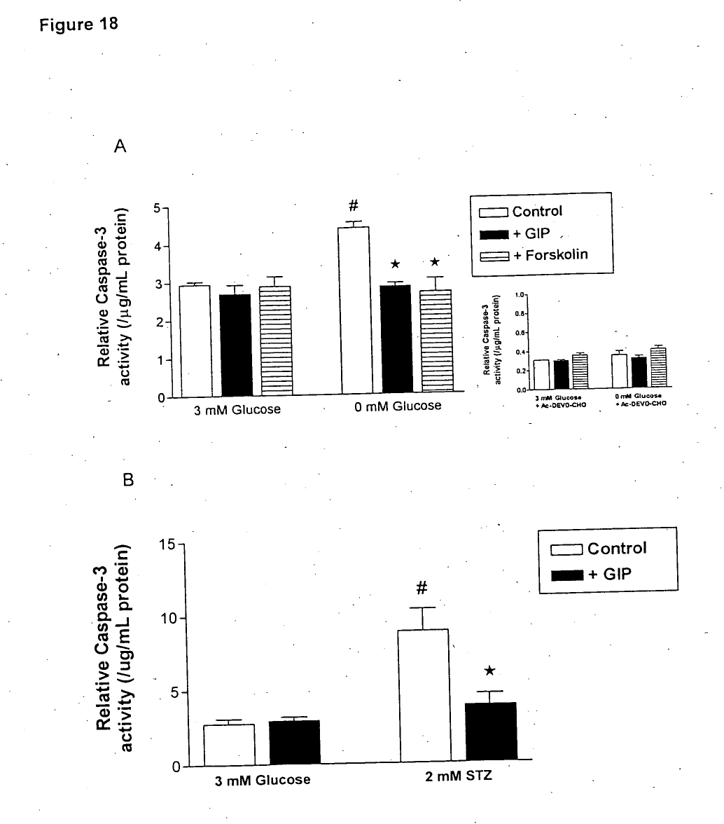

One of the most surprising findings is that the compounds of the present invention have an anti-apoptotic effect on pancreatic β-cells. The anti-apoptotic effect of the compounds of the present invention, including their corresponding pharmaceutically acceptable salts, can be measured employing a caspase-3 activation assay as described in Example 7. The results are shown in FIG. 18A. Caspase-3 activation is a marker for the induction of cellular apoptosis. Based on their receptor binding capabilities and their stimulatory effect on cAMP release, it was found that the compounds of the present invention are able to selectively block activation of caspase-3 in response to glucose withdrawal.

In another in vitro assay, streptozotocin (STZ)-induced β-cell death of INS-1 cells, it has been demonstrated that the compounds of the present invention and including their corresponding pharmaceutically acceptable salts, are able to protect against the pro-apoptotic (caspase-3 activating) effects of STZ completely. The method is described in Example 7. The results are shown in FIG. 18B.

In a further embodiment, the present invention provides pharmaceutical compositions e.g. useful in GIP-receptor binding comprising a pharmaceutically acceptable carrier or diluent and a therapeutically effective amount of a compound of formulas 1-8, or a pharmaceutically acceptable salt thereof.

In still another embodiment, the present invention provides a method for binding or blocking GIP-receptor comprising administering to a mammal in need of such treatment a therapeutically effective amount of a compound of formulas 1-8 above, or a pharmaceutically acceptable salt thereof.

In a further embodiment, the present invention provides a method for treating conditions mediated by GIP-receptor binding comprising administering to a mammal in need of such treatment a therapeutically effective amount of a compound of formulas 1-8 above, or a pharmaceutically acceptable salt thereof.

The present invention also relates to the use of a compound according to the present invention or a pharmaceutically acceptable salt thereof e.g. for the manufacture of a medicament for the prevention or treatment of diseases or conditions associated with GIP-receptor signaling.

In a preferred embodiment, the present invention relates to the use of a compound according to the present invention or a pharmaceutically acceptable salt thereof e.g. for the manufacture of a medicament for the prevention or treatment of diabetes mellitus and obesity.

### EXAMPLES OF THE INVENTION

### Example 1

### Solid-Phase Synthesis of Peptides

The GIP analogs were synthesized with an automated synthesizer SYMPHONY (RAININ) using a modified Fmoc-protocol. Cycles were modified by using double couplings from the 15^{th} amino acid from the C-terminus of the peptide with five-fold excess of Fmoc-amino acids and coupling reagent. The peptide couplings were performed by TBTU/NMM-activation using a 0.23 mmol substituted NovaSyn TGR-resin or the corresponding preloaded Wang-resin at 25 μmol scale. The cleavage from the resin was carried out by a cleavage-cocktail consisting of 94.5% TFA, 2.5% water, 2.5% EDT and 1% TIS.

Analytical and preparative HPLC were performed by using different gradients on the LiChrograph HPLC system of Merck-Hitachi. The gradients were made up from two solvents: (A) 0.1% TFA in H₂O and (B) 0.1% TFA in acetonitrile. Analytical HPLC were performed under the following conditions: solvents were run (1 ml/min) through a 125-4 Nucleosil RP18-column, over a gradient from 5%-50% B over 15 min and then up to 95% B until 20 min, with UV detection (X=220 nm). Purification of the peptides was carried out by preparative HPLC on either a 250-20 Nucleosil 100 RP8-column or a 250-10 LiChrospher 300 RP18-column (flow rate 6 ml/min, 220 nm) under various conditions depending on peptide chain length. For the identification of the peptide analogues, laser desorption mass spectrometry was employed using the HP G2025 MALbI-TOF system of Hewlett-Packard.

### Example 2

### Synthesis of GIP Analogues with a Reduced Peptide Bond

Tyr-Alaψ(CH₂NH)-GIP₃₋₃₀ₐ and Tyr-Alaψ(CH₂NH)-GIP₃₋₁₄ₐ were synthesized by coupling 2 equivalents of Fmoc-Tyr(tBu)ψ(CH2NH)-Glu(tBu)-Gly-OH by TBTU/DIPEA activation and double coupling over 4 hours. The corresponding GIP₅₋₃₀ and GIP₅₋₁₄ fragments were synthesized as described above.

The synthesis of the fully protected tetrapeptide Tyr-Alaψ(CH₂NH)-Glu(tBu)-Gly-OH was carried out on the acid sensitive Sasrin resin in a 0.7 mmol scale by Fmoc-strategy as described in Example 1 using a half-automated peptide synthesizer Labortec (BACHEM). The protected tetrapeptide was cleaved from the resin by 1% TFA. The reduced peptide bond was incorporated via reductive alkylation of the N-terminal deprotected peptide on the sasrin resin with Fmoc-alaninal.

### Example 3

### Determination of DPIV Resistance by MALDI-TOF Mass Spectrometry

The hydrolysis of peptide analogues by purified kidney DPIV was studied as described previously [12]. In brief, peptides were incubated in 0.04 M Tris buffer pH 7.6 and DPIV for up to 24 h. Samples were removed from the incubation mixture and prepared for MALDI-TOF mass spectrometry, as described in Pauly, R. P., Rosche, F., Wermann, M., McIntosh, C. H. S., Pederson, R. A., and Demuth, H. U. Investigation of glucose-dependent insulinotropic polypeptide-(1-42) and glucagon-like peptide-1-(7-36) degradation in vitro by dipeptidyl peptidase IV using matrix-assisted laser desorption/ionization time of flight mass spectrometry—A novel kinetic approach. J Biol Chem 271 (38), 23222-23229.1996.

### Example 4

### In Vitro Studies

Chinese hamster ovary (CHO-K1) cells stably expressing the rat pancreatic islet (wild type) GIP-receptor (wtGIP-R1 cells) were prepared as described previously [19,21]. Cells were cultured in DMEM/F12, supplemented with 10% newborn calf serum, 50 units/ml penicillin G, and 50 μg/ml streptomycin (Culture media and antibiotics from Gibco BRL, Life Technologies). Cells were grown in 75 cm² flasks until 80-90% confluent, when they were split and seeded onto 24 well plates at a density of 50,000 cells/well. Experiments were carried out 48 h later.

Binding Studies

Binding studies using ¹²⁵I-labeled spGIP₁₋₄₂, purified by high performance liquid chromatography (HPLC), were performed essentially as described previously-[21]. wtGIP-R1 Cells (1-5×10⁵/well) were washed twice at 4° C. in binding buffer (BB), consisting of DMEM/F12 (GIBCO), 15 mM HEPES, 0.1% bovine serum albumin (BSA), 1% Trasylol (aprotinin; Bayer), pH 7.4. They were incubated for 12-16 h at 4° C. with ¹²⁵I-spGIP (50,000 cpm) in the presence or absence of unlabeled GIP₁₋₄₂ or analogue. Following incubation, cells were washed twice with ice cold buffer, solubilized with 0.1 M NaOH (1 ml), and transferred to culture tubes for counting of cell-associated radioactivity. Nonspecific binding was defined as that measured in the presence of 1 μM GIP₁₋₄₂ or GIP₁₋₃₀, and specific binding expressed as % of binding in the absence of competitor (% B/Bo).

cAMP Production

Wild type GIP-R1 cells were cultured for 48 h, washed in BB at 37° C., and preincubated for 1 h prior to a 30 min stimulation period with test agents in the presence of 0.5 mM IBMX (Research Biochemicals Intl., Natick, Mass.) [19,21]. With inhibition experiments, cells were incubated with GIP analogues for 15 min prior to a 30 min stimulation with 1 nM shGIP₁₋₄₂. Cells were extracted with 70% ethanol and cAMP levels measured by radioimmunoassay (Biomedical Technologies, Stoughton, Mass.) [19,21]. Data are expressed as fmol/1000 cells or % maximal GIP₁₋₄₂-stimulated cAMP production (inhibition experiments).

### Example 5

### Improvement of Glucose Tolerance after Subcutaneous Administration of Synthetic GIP Analogues to Wistar Rats

Male Wistar rats (250-350 g) were starved overnight (16-18 hours) with free access to drinking water. Whole blood samples were taken from the tail vein of conscious unrestrained rats, for determination of blood glucose (using a hand-held glucometer); plasma was separated by centrifugation (20 min, 12,000 rpm, 4 C) for measurement of plasma insulin concentrations. A basal sample was obtained immediately prior to an oral glucose tolerance test (1 gram glucose/Kg body weight) and intra-scapular subcutaneous injection of peptide analogue (8 nmol/Kg body weight) or saline control (500 microlitre injection volume). Blood samples were taken at t=2, 10, 20, 30, and 60 for insulin determination, and blood glucose was measured at 10 minute intervals. Integrated glucose response was calculated using the trapezoidal algorithm with baseline subtraction.

### Example 6

### GIP Stimulates Cell Proliferation and Promotes Survival of β-(INS-1) Cells

Cell Culture and Reagents

INS-1 cells (clone 832/13) were cultured in 11 mM glucose RPMI (Sigma Laboratories, Natick, Mass., USA) supplemented with 2 mM glutamine, 50 μM β-mercaptoethanol, 10 mM HEPES, 1 mM sodium pyruvate, and 10% fetal bovine serum (Cansera, Rexdale, Ont., Canada). Prior to experiments, cells were harvested into either 6-well (2×10⁶ cells/well; Becton Dickinson, Licoln Park, N.J., USA), 24-well (5×10⁵ cells/well), or 96-well (5×10⁴ cells/well) plates. Cell passages 45-60 were used.

GIP-Receptor Characterization Studies; Competitive Binding, cAMP, and Insulin Release

Synthetic porcine GIP (5 μg) was iodinated by the chloramine-T method, and the ¹²⁵I-GIP was further purified by reverse phase high performance liquid chromatography to a specific activity of 250-300 μCi/μg. Competitive binding analyses were performed as described in Example 4. For cAMP studies, cells were washed twice and then stimulated for 30 minutes with GIP in the presence of the phophodiesterase inhibitor 3-isobutyl-1-methylxanthine (0.5 mM IBMX; RBI/Sigma, Natick, Mass., USA). Following stimulation, reactions were stopped, and cells lysed, in 70% ice-cold ethanol, cellular debris removed by centrifugation, and cAMP subsequently quantified by radioimmunoassy (RIA) (Biomedical Technologies Inc., Stoughton, Mass., USA). All insulin release experiments were performed over 60 minutes, in the absence of IBMX, and insulin secreted into the media was quantified by RIA.

Since GIP-receptors in the INS-1 clone 832/13 cell line had not been previously characterized, binding, adenylyl cyclase stimulation and insulin secretory responses to GIP were initially studied. Cells expressed receptors at a density of 1571±289 binding sites/cell (n=3) with an IC₅₀ for binding of 21.1±2.49 nM (n=3) and a K_{D}=106.2±4.3 fmol (n=3); cAMP production was stimulated by GIP with an EC₅₀ of 4.70±1.81 nM (n=4)); 5.5 mM glucose stimulated insulin secretion was potentiated by 10 nM GIP (1.63±0.183% total insulin secreted for 5.5 mM glucose vs. 2.44±0.29% total insulin secreted (p<0.05, n=3)).

Cell Quantification

Cells were seeded into 96-well plates (5×10⁴ cells/well) prior to experimentation. After establishing metabolic quiescence in the absence of serum for 24 h, cells were cultured in low glucose media (RPMI with 0.1% BSA) with agonists (glucose, glucose+GIP/GLP-1/GH) for an additional 24 h. Thereafter, cells were washed with KRBH (115 mM NaCl, 4.7 mM KCl, 1.2 mM KH₂PO₄, 10 mM NaHCO₃, 1.28 mM CaCl₂, 1.2 mM MgSO₄ containing 10 mM HEPES and 0.1% bovine serum albumin, pH 7.4) and frozen at −70° C. until assayed. Cells were quantified using the CYQUANT™ assay system (Molecular Probes, Eugene, Oreg., USA) according to the manufacturers' protocol. Final cell numbers were always greater than the initial number plated in assessing cellular proliferation.

Cell survival was assessed in the presence of prolonged glucose deprivation. 24 h after glucose deprivation (RPMI with 0.1% BSA), GIP or forskolin were added for an additional 24 h, and cell number was quantified. Final cell numbers were always less than the initial number plated in assessing cell survival.

GIP Potentiates Glucose Dependent β-Cell Proliferation

The INS-1 cell line has been extensively investigated previously as a cellular model for β-cell proliferation (Hugl S R, White M F, Rhodes C J 1998: Insulin-like growth factor 1 (IGF-1)-stimulated pancreatic beta-cell growth is glucose-dependent. J. Biol. Chem. 273: 17771-17779; Dickson L M, Linghor M K, McCuaig J, Hugl S R, Snow L, Kahn B B, Myers Jr. M G, Rhodes C J (2001), Differential activation of protein kinase B and p70S6K by glucose and insulin-like growth factor 1 in pancreatic beta cells (INS-1). J. Biol. Chem. 276:21110-21120). GIP was found to potentiate 11 mM glucose mediated β-cell proliferation (FIG. 15A) to levels comparable to those obtained with GH (158±16% of growth in the presence of 5.5 mM glucose for 100 nM GIP; 158±9% for 10 nM GH (n=3-5)). In a separate experiment (FIG. 16B), 100 nM GIP stimulated cell growth to 131±7% of that measured in the presence of 5.5 mM glucose, similar to the proliferative responses to 100 nM GLP-1 (129±4%; n=4).

GIP Reverses the Detrimental Effect of 0 mM Glucose

While determining the glucose-dependence of these growth promotive effects, it was observed that GIP was capable of reversing the detrimental effects of 0 mM glucose media on cellular survival. Incubation of cells in the presence of 0 mM glucose media for 48 h resulted in approximately 50% cell death (FIG. 16A). Surprisingly, 91±10% of the cells plated remained viable when the media was supplemented with 100 nM GIP after 24 h. These cell survival effects of GIP were found to be concentration-dependent with an EC₅₀ value of 1.24±0.48 nM GIP (n=4; FIG. 16B).

GIP has a Protective Effect Against Wortmannin-Induced Cell Death

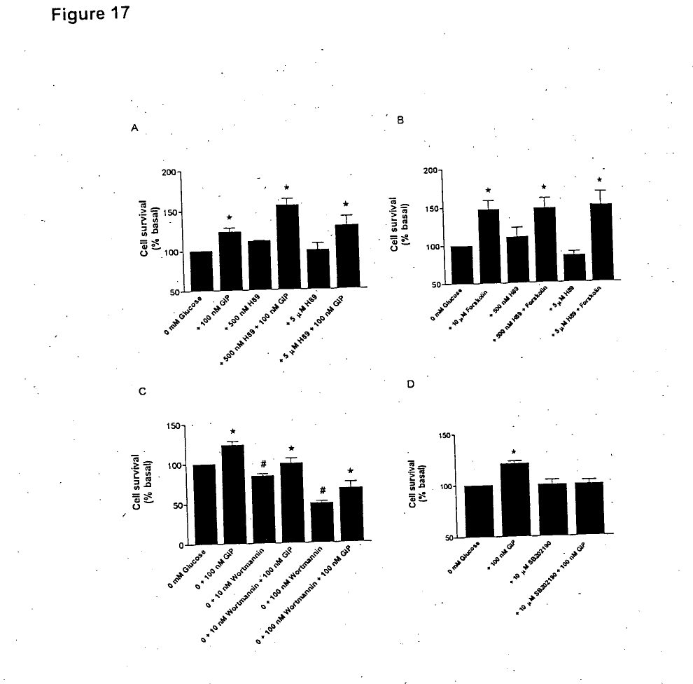

In order to establish which intracellular signaling pathways were involved in the GIP-induced cell survival, studies were performed with pharmacological inhibitors used at concentrations shown to exhibit selectivity for candidate protein kinases (FIG. 17). Stimulation of adenylyl cyclase with forskolin mimicked the effects of GIP on cell survival, but the lack of effect of H89 (FIGS. 18A and B) indicates a PKA-independent mode of action. Neither of the Mek1/2 inhibitors PD98059 (50 and 100 μM) nor U0126 (10 μM) blocked the effects of GIP on cell survival (n=3). The ability of GIP to promote cell survival was further supported by studies on the effect of the PI3Kinase-PKB pathway inhibitor, wortmannin (FIG. 17C). Since wortmannin alone promoted cell loss it was not possible to determine whether GIP activates the PI3Kinase-PKB pathway. However, cells were partially protected against wortmannin-induced cell loss by GIP treatment (n=3, p<0.05). The only compound tested that influenced GIP-mediated cell survival was the inhibitor SB202190 (FIG. 17D), indicating that GIP can act via p38 MAPK.

### Example 7

### GIP has an Anti-Apoptotic Effect

Caspase-3 Activity

INS-1 cells (clone 832/13) seeded into 6-well plates were serum starved for 12-24 h and subjected to glucose deprivation (RPMI with 0.1% BSA) or treatment with 2 mM streptozotocin (STZ). GIP and GLP-1 were added 10 min prior to STZ and for 30 min during STZ. Following treatment, caspase-3 activity was determined after 2, 6, or 24 h according to the manufacturers' protocol (Molecular Probes, Eugene, Oreg., USA). Caspase-3 activity/well was corrected for total protein content using the BCA protein assay (Pierce, Roxford, II, USA).

Caspase-3 activation is a marker for the induction of cellular apoptosis. To establish whether the cell survival effects of GIP were due to anti-apoptotic actions of the polypeptide, activation of caspase-3 induced by glucose deprivation was studied. FIG. 18A illustrates that 0 mM glucose promoted apoptosis by 6 h (not by 2 h; data not shown), and that this effect was completely reversed by addition of GIP or forskolin. The conclusion that GIP selectively blocked activation of caspase-3 in response to glucose withdrawal was confirmed by the demonstration that the specific aldehyde inhibitor of caspase-3, Ac-DEVD-CHO, completely blocked low glucose activation (FIG. 19A).

STZ-Induced Cell Death

The ability of GIP to protect against streptozotocin (STZ)-induced β-cell death was studied. When added 10 minutes prior to, and during, a 30 minute STZ exposure, GIP was able to protect against the pro-apoptotic (caspase-3 activating) effects of STZ completely (FIG. 18B).

1. A novel GIP analogue which codes an amino acid sequence shown by formula 1:
(SEQ ID NO: 49)

| | |
|---|---|
| Tyr-A-B-Gly-Thr-Phe-Ile-Ser-Asp-Tyr-Ser-Ile- | (1) |
| Ala-Met | |

wherein A and B are amino acid residues including D-amino acid residues, N-methylated amino acid residues and any other non-proteinogenic amino acid residues or a pharmaceutically acceptable salt thereof, excluding the sequence of native GIP (1-14).

2. A novel GIP analogue according to claim 1, wherein the N-terminus of the tyrosine residue in position 1 can be modified by alkylation, sulphonylation, glycation, homoserine formation, pyroglutamic acid formation, disulphide bond formation, deamidation of asparagine or glutamine residues, methylation, t-butylation, t-butyloxycarbonylation, 4-methylbenzylation, thioanysilation, thiocresylation, bencyloxymethylation, 4-nitrophenylation, bencyloxycarbonylation, 2-nitrobencoylation, 2-nitrosulphenylation, 4-toluenesulphonylation, pentafluorophenylation, diphenylmethylation, 2-chlorobenzyloxycarbonylation, 2,4,5-trichlorophenylation, 2-bromobenzyloxycarbonylation, 9-fluorenylmethyloxycarbonylation, triphenylmethylation, 2,2,5,7,8,-pentamethylchroman-6-sulphonylation, hydroxylation, oxidation of methionine, formylation, acetylation, anisylation, bencylation, bencoylation, trifluoroacetylation, carboxylation of aspartic acid or glutamic acid, phosphorylation, sulphation, cysteinylation, glycolysation with pentoses, deoxyhexoses, hexosamines, hexoses or N-acetylhexosamines, farnesylation, myristolysation, biotinylation, palmitoylation, stearoylation, geranylgeranylation, glutathionylation, 5′-adenosylation, ADP-ribosylation, modification with N-glycolylneuraminic acid, N-acetylneuraminic acid, pyridoxal phosphate, lipoic acid, 4′-phosphopantetheine, and N-hydroxysuccinimide.

3. A novel GIP analogue according to claim 1, wherein the peptide is modified by the introduction of at least one 6-amino fatty acid acylated lysine in any amino acid position.

4. A compound according to claim 1 having the amino acid sequence:
(SEQ ID NO: 21)
Tyr-(D-Ala)-Glu-Gly-Thr-Phe-Ile-Ser-Asp-Tyr-Ser-
Ile-Ala-Met.

5. A compound according to claim 1 having the amino acid sequence:
(SEQ ID NO: 20)
Tyr-Ala-Pro-Gly-Thr-Phe-Ile-Ser-Asp-Tyr-Ser-Ile-
Ala-Met.

6. GIP analogues having the amino acid sequences and comprising a reduced peptide bond:
(SEQ ID NO: 19)
Tyr-Ala-Ψ(CH₂NH₂)-Glu-Gly-Thr-Phe-Ile-Ser-Asp-Tyr-
Ser-Ile-Ala-Met;
(SEQ ID NO: 11)
Tyr-Ala-Ψ(CH₂NH)-Glu-Gly-Thr-Phe-Ile-Ser-Asp-Tyr-
Ser-Ile-Ala-Met-Asp-Lys-Ile-His-Gln-Gln-Asp-Phe-
Val-Asn-Trp-Leu-Leu-Ala-Gln-Lys;
and
pharmaceutically acceptable salts thereof.

7. A GIP analogue having the amino acid sequence:
(SEQ ID NO: 51)
Tyr-Ala-Glu-Gly-Thr-Phe-Ile-Ser-Asp-Tyr-Ser-Ile-
Tyr-Met
or a pharmaceutically acceptable salt thereof.

8. GIP analogues having the amino acid sequences:
(SEQ ID NO: 52)
Ala-Ala-Glu-Gly-Thr-Phe-Ile-Ser-Asp-Tyr-Ser-Ile-
Ala-Met,
(SEQ ID NO: 53)
Tyr-Ala-Ala-Gly-Thr-Phe-Ile-Ser-Asp-Tyr-Ser-Ile-
Ala-Met;
(SEQ ID NO: 54)
Tyr-Ala-Glu-Ala-Thr-Phe-Ile-Ser-Asp-Tyr-Ser-Ile-
Ala-Met;
(SEQ ID NO: 55)
Tyr-Ala-Glu-Gly-Ala-Phe-Ile-Ser-Asp-Tyr-Ser-Ile-
Ala-Met;
(SEQ ID NO: 56)
Tyr-Ala-Glu-Gly-Thr-Ala-Ile-Ser-Asp-Tyr-Ser-Ile-
Ala-Met;
(SEQ ID NO: 57)
Tyr-Ala-Glu-Gly-Thr-Phe-Ala-Ser-Asp-Tyr-Ser-Ile-
Ala-Met;
(SEQ ID NO: 58)
Tyr-Ala-Glu-Gly-Thr-Phe-Ile-Ala-Asp-Tyr-Ser-Ile-
Ala-Met;
(SEQ ID NO: 59)
Tyr-Ala-Glu-Gly-Thr-Phe-Ile-Ser-Ala-Tyr-Ser-Ile-
Ala-Met;
(SEQ ID NO: 60)
Tyr-Ala-Glu-Gly-Thr-Phe-Ile-Ser-Asp-Ala-Ser-Ile-
Ala-Met;
(SEQ ID NO: 61)
Tyr-Ala-Glu-Gly-Thr-Phe-Ile-Ser-Asp-Tyr-Ala-Ile-
Ala-Met;
(SEQ ID NO: 62)
Tyr-Ala-Glu-Gly-Thr-Phe-Ile-Ser-Asp-Tyr-Ser-Ala-
Ala-Met;
(SEQ ID NO: 63)
Tyr-Ala-Glu-Gly-Thr-Phe-Ile-Ser-Asp-Tyr-Ser-Ile-
Ala-Ala;
and
or a pharmaceutically acceptable salt thereof.

9. GIP analogues having the amino acid sequence and comprising linker peptides:
(SEQ ID NO: 64)
Tyr-A-B-Gly-Thr-Phe-C-Gln-Gln-Asp-Phe-Val-Asn-Trp-
Leu-Leu-Ala-Gln-Lys-Gly-Lys-Lys-Asn-Asp-Trp-Lys-
His-Asn-Ile-Thr-Gln;
wherein C is
a) not used,
b) a linker peptide comprising 4 amino acid residues selected from the group consisting of amino acid residues, D-amino acids and non-proteinogenic amino acids, is allowed and within the scope of the present invention,

| | | |
|---|---|---|
| c) | Glu-Lys-Glu-Lys, | (SEQ ID NO: 65) |
| d) | Ala-Ala-Ala-Ala, | (SEQ ID NO: 66) |

e) a linker peptide comprising 12 amino acid residues selected from the group consisting of amino acid residues, D-amino acids and non-proteinogenic amino acids,
f) Glu-Lys-Glu-Glu-Lys-Glu-Lys-Glu-Glu-Lys-Glu-Lys, (SEQ ID NO: 67)
g) 6-Ahxₙ (6-aminohexanoic acid) with n=1-3, or
h) Omega-amino fatty acids (saturated and unsaturated) of ω-NH₂—(CHx)n-COOH with n=10-34; and
wherein A and B are amino acid residues, D-amino acid residues, N-methylated amino acid residues or any other non-proteinogenic amino acid residues; and
pharmaceutically acceptable salts thereof.

10. A GIP analogue according to claim 9, wherein the N-terminus of the tyrosine residue in position 1 can be modified by alkylation, sulphonylation, glycation, homoserine formation, pyroglutamic acid formation, disulphide bond formation, deamidation of asparagine or glutamine residues, methylation, t-butylation, t-butyloxycarbonylation, 4-methylbenzylation, thioanysilation, thiocresylation, bencyloxymethylation, 4-nitrophenylation, bencyloxycarbonylation, 2-nitrobencoylation, 2-nitrosulphenylation, 4-toluenesulphonylation, pentafluorophenylation, diphenylmethylation, 2-chlorobenzyloxycarbonylation, 2,4,5-trichlorophenylation, 2-bromobenzyloxycarbonylation, 9-fluorenylmethyloxycarbonylation, triphenylmethylation, 2,2,5,7,8,-pentamethylchroman-6-sulphonylation, hydroxylation, oxidation of methionine, formylation, acetylation, anisylation, bencylation, bencoylation, trifluoroacetylation, carboxylation of aspartic acid or glutamic acid, phosphorylation, sulphation, cysteinylation, glycolysation with pentoses, deoxyhexoses, hexosamines, hexoses or N-acetylhexosamines, farnesylation, myristolysation, biotinylation, palmitoylation, stearoylation, geranylgeranylation, glutathionylation, 5′-adenosylation, ADP-ribosylation, modification with N-glycolylneuraminic acid, N-acetylneuraminic acid, pyridoxal phosphate, lipoic acid, 4′-phosphopantetheine, and N-hydroxysuccinimide.

11. A GIP analogue according to claim 10, wherein the peptide is modified by the introduction of at least one ε-amino fatty acid acylated lysine in any amino acid position.

12. A novel GIP analogue according to claim 10, wherein the peptide is modified by introduction of a reduced peptide bond or any other modification of the peptide bond between A and B.

13. Novel GIP analogues having the amino acid sequence and comprising linker peptides:
(SEQ ID NO: 68)
Tyr-A-B-Gly-Thr-Phe-Ile-Ser-Asp-Tyr-Ser-Ile-Ala-
Met-D-Gln-Gln-Asp-Phe-Val-Asn-Trp-Leu-Leu-Ala-Gln-
Lys-Gly-Lys-Lys-Asn-Asp-Trp-Lys-His-Asn-Ile-Thr-
Gln
wherein D is
a) unused,
b) a linker peptide comprising 4 amino acid residues selected from the group consisting of amino acid residues, D-amino acids and non-proteinogenic amino acids,
c) Ala-Ala-Ala-Ala, (SEQ ID NO: 66)
d) Glu-Lys-Glu-Lys, (SEQ ID NO: 65)
e) 6-Ahxₙ (6-aminohexanoic acid) with n=1-3, or
f) an omega-amino fatty acid (saturated and unsaturated) of ω-NH₂—(CHx)n-COOH with n=10-34; and
wherein A and B are amino acid residues, D-amino acid residues, N-methylated amino acid residues or any other non-proteinogenic amino acid residues; and
pharmaceutically acceptable salts thereof.

14. A GIP analogue according to claim 13, wherein the N-terminus of the tyrosine residue in position 1 can be modified by alkylation, acetylation or glycation.

15. A GIP analogue according to claim 13, wherein the peptide is modified by the introduction of at least one ε-amino fatty acid acylated lysine in any amino acid position.

16. A GIP analogue according to claim 13, wherein the peptide is modified by introduction of a reduced peptide bond or other modification of the peptide bond between A and B.

17. GIP analogues having the amino acid sequences and comprising a phosphorylated seryl residue:
(SEQ ID NO: 69)
Tyr-[Ser(P)]-Glu-Gly-Thr-Phe-Ile-Ser-Asp-Tyr-Ser-
Ile-Ala-Met,
(SEQ ID NO: 70)
Tyr-[Ser(P)]-Glu-Gly-Thr-Phe-Ile-Ser-Asp-Tyr-Ser-
Ile-Ala-Met-Asp-Lys-Ile-His-Gln-Gln-Asp-Phe-Val-
Asn-Trp-Leu-Leu-Ala-Gln-Lys,
(SEQ ID NO: 71)
Tyr-[Ser(P)]-Glu-Gly-Thr-Phe-Ile-Ser-Asp-Tyr-Ser-
Ile-Ala-Met-Asp-Lys-Ile-His-Gln-Gln-Asp-Phe-Val-
Asn-Trp-Leu-Leu-Ala-Gln-Lys-Gly-Lys-Lys-Asn-Asp-
Trp-Lys-His-Asn-Ile-Thr-Gln;
and
pharmaceutically acceptable salts thereof.

18. A compound having the amino acid sequence: 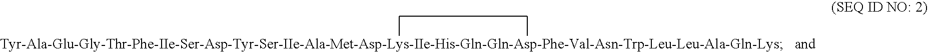
a pharmaceutically acceptable salt thereof.

19. A compound according to claim 1 in free carboxylic acid form or a pharmaceutically acceptable salt thereof.

20. A compound according to claim 1 in amid form or a pharmaceutically acceptable salt thereof.

21. A compound according to claim 1 characterized in that the compound is resistant to the degradation by dipeptidyl peptidase IV or dipeptidyl peptidase IV-like enzyme activity.

22. A compound according to claim 1 characterized in that the compound is a GIP-receptor agonist.

23. A compound according to claim 1 characterized in that the compound is a GIP-receptor antagonist.

24. A compound according to claim 1 characterized in that the compound potentiates cyclic AMP production.

25. A compound according to claim 1 characterized in that the compound blocks the activation of caspase-3.

26. A pharmaceutical composition comprising a pharmaceutically acceptable carrier or diluent and a therapeutically effective amount of a compound according to claim 1, or a pharmaceutically acceptable acid addition salt thereof.

27. Use of a compound according to claim 1 or a pharmaceutically acceptable acid addition salt thereof for the manufacture of a medicament for GIP-receptor binding for the prevention or treatment of diseases or conditions related to impaired binding of GIP-receptor analogues.

28. Use according to claim 27 for the manufacture of a medicament for the prevention or treatment of β-cell apoptosis.

29. Use according to claim 27 for the manufacture of a medicament for the potentiation of glucose dependent proliferation of pancreatic β-cells

30. Use according to claim 27 for the manufacture of a medicament for the treatment of non-insulin-dependent diabetes mellitus and obesity.

31. A method for treating conditions mediated by GIP-receptor binding comprising administering to a mammal in need of such treatment a therapeutically effective amount of a compound according to claim 1.

32. A method for lowering elevated blood glucose levels in mammals resulting from food intake comprising administering a therapeutically effective amount of at least one compound according to claim 1.


SEQUENCE LISTING (82 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 30 aa, Artificial Sequence
  Description of Artificial Sequence Synthetic GIP analog
YAEGTFISDYSIAMDKIHQQDFVNWLLAQK

SEQ ID NO: 3 - protein, 40 aa, Artificial Sequence
  Description of Artificial Sequence Synthetic GIP analog
EGTFISDYSIAMDKIHQQDFVNWLLAQKGKKNDWKHNITQ

SEQ ID NO: 4 - protein, 30 aa, Artificial Sequence; identical to SEQ ID NO: 2
  Description of Artificial Sequence Synthetic GIP analog

SEQ ID NO: 5 - protein, 30 aa, Artificial Sequence; identical to SEQ ID NO: 2
  Description of Artificial Sequence Synthetic GIP analog

SEQ ID NO: 6 - protein, 30 aa, Artificial Sequence; identical to SEQ ID NO: 2
  Description of Artificial Sequence Synthetic GIP analog

SEQ ID NO: 7 - protein, 30 aa, Artificial Sequence
  Description of Artificial Sequence Synthetic GIP analog
YAPGTFISDYSIAMDKIHQQDFVNWLLAQK

SEQ ID NO: 8 - protein, 30 aa, Artificial Sequence
  Description of Artificial Sequence Synthetic GIP analog
YSEGTFISDYSIAMDKIHQQDFVNWLLAQK

SEQ ID NO: 9 - protein, 30 aa, Artificial Sequence
  Description of Artificial Sequence Synthetic GIP analog
YVEGTFISDYSIAMDKIHQQDFVNWLLAQK

SEQ ID NO: 10 - protein, 30 aa, Artificial Sequence
  Description of Artificial Sequence Synthetic GIP analog
YGEGTFISDYSIAMDKIHQQDFVNWLLAQK

SEQ ID NO: 11 - protein, 30 aa, Artificial Sequence; identical to SEQ ID NO: 2
  Description of Artificial Sequence Synthetic GIP analog

SEQ ID NO: 12 - protein, 6 aa, Artificial Sequence
  Description of Artificial Sequence Synthetic GIP analog
YAEGTF

SEQ ID NO: 13 - protein, 6 aa, Artificial Sequence
  Description of Artificial Sequence Synthetic GIP analog
YAEGTF

SEQ ID NO: 14 - protein, 6 aa, Artificial Sequence
  Description of Artificial Sequence Synthetic GIP analog
YGEGTF

SEQ ID NO: 15 - protein, 6 aa, Artificial Sequence
  Description of Artificial Sequence Synthetic GIP analog
YSEGTF

SEQ ID NO: 16 - protein, 6 aa, Artificial Sequence
  Description of Artificial Sequence Synthetic GIP analog
YPEGTF

SEQ ID NO: 17 - protein, 6 aa, Artificial Sequence
  Description of Artificial Sequence Synthetic GIP analog
YVEGTF

SEQ ID NO: 18 - protein, 6 aa, Artificial Sequence
  Description of Artificial Sequence Synthetic GIP analog
YAPGTF

SEQ ID NO: 19 - protein, 14 aa, Artificial Sequence
  Description of Artificial Sequence Synthetic GIP analog
YAEGTFISDYSIAM

SEQ ID NO: 20 - protein, 14 aa, Artificial Sequence
  Description of Artificial Sequence Synthetic GIP analog
YAPGTFISDYSIAM

SEQ ID NO: 21 - protein, 14 aa, Artificial Sequence
  Description of Artificial Sequence Synthetic GIP analog
YAEGTFISDYSIAM

SEQ ID NO: 22 - protein, 13 aa, Artificial Sequence
  Description of Artificial Sequence Synthetic GIP analog
YAEGTFISDYSIA

SEQ ID NO: 23 - protein, 15 aa, Artificial Sequence
  Description of Artificial Sequence Synthetic GIP analog
YAEGTFISDYSIAMD

SEQ ID NO: 24 - protein, 16 aa, Artificial Sequence
  Description of Artificial Sequence Synthetic GIP analog
DKIHQQDFVNWLLAQK

SEQ ID NO: 25 - protein, 14 aa, Artificial Sequence
  Description of Artificial Sequence Synthetic GIP analog
IHQQDFVNWLLAQK

SEQ ID NO: 26 - protein, 12 aa, Artificial Sequence
  Description of Artificial Sequence Synthetic GIP analog
QQDFVNWLLAQK

SEQ ID NO: 27 - protein, 24 aa, Artificial Sequence
  Description of Artificial Sequence Synthetic GIP analog
ISDYSIAMDKIHQQDFVNWLLAQK

SEQ ID NO: 28 - protein, 7 aa, Artificial Sequence
  Description of Artificial Sequence Synthetic GIP analog
YAEGTFI

SEQ ID NO: 29 - protein, 14 aa, Artificial Sequence
  Description of Artificial Sequence Synthetic GIP analog
YAEGTFISDYSIAM

SEQ ID NO: 30 - protein, 28 aa, Artificial Sequence
  Description of Artificial Sequence Synthetic GIP analog
DKIHQQDFVNWLLAQKGKKNDWKHNITQ

SEQ ID NO: 31 - protein, 15 aa, Artificial Sequence
  Description of Artificial Sequence Synthetic GIP analog
KIHQQDFVNWLLAQK

SEQ ID NO: 32 - protein, 18 aa, Artificial Sequence
  Description of Artificial Sequence Synthetic GIP analog
YAEGTFQQDFVNWLLAQK

SEQ ID NO: 33 - protein, 22 aa, Artificial Sequence
  Description of Artificial Sequence Synthetic GIP analog
YAEGTFAAAAQQDFVNWLLAQK

SEQ ID NO: 34 - protein, 22 aa, Artificial Sequence
  Description of Artificial Sequence Synthetic GIP analog
YAEGTFEKEKQQDFVNWLLAQK

SEQ ID NO: 35 - protein, 29 aa, Artificial Sequence
  Description of Artificial Sequence Synthetic GIP analog
YAEGTFEKEEKEKEEKEQQDFVNWLLAQK

SEQ ID NO: 36 - protein, 19 aa, Artificial Sequence
  Description of Artificial Sequence Synthetic GIP analog
YAEGTFXQQDFVNWLLAQK

SEQ ID NO: 37 - protein, 20 aa, Artificial Sequence
  Description of Artificial Sequence Synthetic GIP analog
YAEGTFXXQQDFVNWLLAQK

SEQ ID NO: 38 - protein, 21 aa, Artificial Sequence
  Description of Artificial Sequence Synthetic GIP analog
YAEGTFXXXQQDFVNWLLAQK

SEQ ID NO: 39 - protein, 26 aa, Artificial Sequence
  Description of Artificial Sequence Synthetic GIP analog
YAEGTFISDYSIAMQQDFVNWLLAQK

SEQ ID NO: 40 - protein, 30 aa, Artificial Sequence
  Description of Artificial Sequence Synthetic GIP analog
YAEGTFISDYSIAMAAAAQQDFVNWLLAQK

SEQ ID NO: 41 - protein, 30 aa, Artificial Sequence
  Description of Artificial Sequence Synthetic GIP analog
YAEGTFISDYSIAMEKEKQQDFVNWLLAQK

SEQ ID NO: 42 - protein, 27 aa, Artificial Sequence
  Description of Artificial Sequence Synthetic GIP analog
YAEGTFISDYSIAMXQQDFVNWLLAQK

SEQ ID NO: 43 - protein, 28 aa, Artificial Sequence
  Description of Artificial Sequence Synthetic GIP analog
YAEGTFISDYSIAMXXQQDFVNWLLAQK

SEQ ID NO: 44 - protein, 29 aa, Artificial Sequence
  Description of Artificial Sequence Synthetic GIP analog
YAEGTFISDYSIAMXXXQQDFVNWLLAQK

SEQ ID NO: 45 - protein, 30 aa, Artificial Sequence; identical to SEQ ID NO: 2
  Description of Artificial Sequence Synthetic GIP analog

SEQ ID NO: 46 - protein, 25 aa, Artificial Sequence
  Description of Artificial Sequence Synthetic GIP analog
FISDYSIAMDKIHQQDFVNWLLAQK

SEQ ID NO: 47 - protein, 43 aa, Artificial Sequence
  Description of Artificial Sequence Synthetic GIP formula peptide
YAEGTFISDYSIAXDKIHQQDFVNWLLAQKXXXXXXXXXXXXX

SEQ ID NO: 48 - protein, 12 aa, Artificial Sequence
  Description of Artificial Sequence Synthetic peptide
GKKNDWKHNITQ

SEQ ID NO: 49 - protein, 14 aa, Artificial Sequence
  Description of Artificial Sequence Synthetic GIP formula peptide
YXXGTFISDYSIAM

SEQ ID NO: 50 - protein, 14 aa, Artificial Sequence
  Description of Artificial Sequence Synthetic GIP analog
YSEGTFISDYSIAM

SEQ ID NO: 51 - protein, 14 aa, Artificial Sequence
  Description of Artificial Sequence Synthetic GIP analog
YAEGTFISDYSIYM

SEQ ID NO: 52 - protein, 14 aa, Artificial Sequence
  Description of Artificial Sequence Synthetic GIP analog
AAEGTFISDYSIAM

SEQ ID NO: 53 - protein, 14 aa, Artificial Sequence
  Description of Artificial Sequence Synthetic GIP analog
YAAGTFISDYSIAM

SEQ ID NO: 54 - protein, 14 aa, Artificial Sequence
  Description of Artificial Sequence Synthetic GIP analog
YAEATFISDYSIAM

SEQ ID NO: 55 - protein, 14 aa, Artificial Sequence
  Description of Artificial Sequence Synthetic GIP analog
YAEGAFISDYSIAM

SEQ ID NO: 56 - protein, 14 aa, Artificial Sequence
  Description of Artificial Sequence Synthetic GIP analog
YAEGTAISDYSIAM

SEQ ID NO: 57 - protein, 14 aa, Artificial Sequence
  Description of Artificial Sequence Synthetic GIP analog
YAEGTFASDYSIAM

SEQ ID NO: 58 - protein, 14 aa, Artificial Sequence
  Description of Artificial Sequence Synthetic GIP analog
YAEGTFIADYSIAM

SEQ ID NO: 59 - protein, 14 aa, Artificial Sequence
  Description of Artificial Sequence Synthetic GIP analog
YAEGTFISAYSIAM

SEQ ID NO: 60 - protein, 14 aa, Artificial Sequence
  Description of Artificial Sequence Synthetic GIP analog
YAEGTFISDASIAM

SEQ ID NO: 61 - protein, 14 aa, Artificial Sequence
  Description of Artificial Sequence Synthetic GIP analog
YAEGTFISDYAIAM

SEQ ID NO: 62 - protein, 14 aa, Artificial Sequence
  Description of Artificial Sequence Synthetic GIP analog
YAEGTFISDYSAAM

SEQ ID NO: 63 - protein, 14 aa, Artificial Sequence
  Description of Artificial Sequence Synthetic GIP analog
YAEGTFISDYSIAA

SEQ ID NO: 64 - protein, 51 aa, Artificial Sequence
  Description of Artificial Sequence Synthetic GIP formula peptide
YXXGTFXXXXXXXXXXXXXXXXXXXXXQQDFVNWLLAQKGKKNDWKHNITQ

SEQ ID NO: 65 - protein, 4 aa, Artificial Sequence
  Description of Artificial Sequence Synthetic linker peptide
EKEK

SEQ ID NO: 66 - protein, 4 aa, Artificial Sequence
  Description of Artificial Sequence Synthetic linker peptide
AAAA

SEQ ID NO: 67 - protein, 12 aa, Artificial Sequence
  Description of Artificial Sequence Synthetic peptide
EKEEKEKEEKEK

SEQ ID NO: 68 - protein, 59 aa, Artificial Sequence
  Description of Artificial Sequence Synthetic GIP formula peptide
YXXGTFISDYSIAMXXXXXXXXXXXXXXXXXXXXXQQDFVNWLLAQKGKKNDWKHNITQ

SEQ ID NO: 69 - protein, 14 aa, Artificial Sequence
  Description of Artificial Sequence Synthetic GIP analog
YSEGTFISDYSIAM

SEQ ID NO: 70 - protein, 30 aa, Artificial Sequence; identical to SEQ ID NO: 8
  Description of Artificial Sequence Synthetic GIP analog

SEQ ID NO: 71 - protein, 42 aa, Artificial Sequence
  Description of Artificial Sequence Synthetic GIP analog
YSEGTFISDYSIAMDKIHQQDFVNWLLAQKGKKNDWKHNITQ

SEQ ID NO: 72 - protein, 30 aa, Artificial Sequence; identical to SEQ ID NO: 2
  Description of Artificial Sequence Synthetic GIP analog

SEQ ID NO: 73 - protein, 4 aa, Artificial Sequence
  Description of Artificial Sequence Synthetic tetrapeptide
YAEG

SEQ ID NO: 74 - protein, 14 aa, Artificial Sequence
  Description of Artificial Sequence Synthetic GIP analog
YAEGTFISDYSIAM

SEQ ID NO: 75 - protein, 42 aa, Artificial Sequence; identical to SEQ ID NO: 1
  Description of Artificial Sequence Synthetic GIP analog

SEQ ID NO: 76 - protein, 14 aa, Artificial Sequence
  Description of Artificial Sequence Synthetic GIP analog
TAEGTFISDYSIAM

SEQ ID NO: 77 - protein, 42 aa, Artificial Sequence; identical to SEQ ID NO: 71
  Description of Artificial Sequence Synthetic GIP analog

SEQ ID NO: 78 - protein, 42 aa, Artificial Sequence; identical to SEQ ID NO: 71
  Description of Artificial Sequence Synthetic GIP analog

SEQ ID NO: 79 - protein, 420 aa, Artificial Sequence
  Description of Artificial Sequence Synthetic GIP analog
YSEGTFISDYSIAMDKIHQQDFVNWLLAQKGKKNDWKHNITQYSEGTFISDYSIAMDKIHQQDFVNWLLAQKGKKNDWKHNITQYSEGTFISDYSIAMDKIHQQDFVNWLLAQKGKKNDWKHNITQYSEGTFISDYSIAMDKIHQQDFVNWLLAQKGKKNDWKHNITQYSEGTFISDYSIAMDKIHQQDFVNWLLAQKGKKNDWKHNITQYSEGTFISDYSIAMDKIHQQDFVNWLLAQKGKKNDWKHNITQYSEGTFISDYSIAMDKIHQQDFVNWLLAQKGKKNDWKHNITQYSEGTFISDYSIAMDKIHQQDFVNWLLAQKGKKNDWKHNITQYSEGTFISDYSIAMDKIHQQDFVNWLLAQKGKKNDWKHNITQYSEGTFISDYSIAMDKIHQQDFVNWLLAQKGKKNDWKHNITQ

SEQ ID NO: 80 - protein, 14 aa, Artificial Sequence
  Description of Artificial Sequence Synthetic GIP analog
TAEGTFISDYSIAM

SEQ ID NO: 81 - protein, 30 aa, Artificial Sequence
  Description of Artificial Sequence Synthetic GIP analog
TAEGTFISDYSIAMDKIHQQDFVNWLLAQK

SEQ ID NO: 82 - protein, 42 aa, Artificial Sequence; identical to SEQ ID NO: 1
  Description of Artificial Sequence Synthetic GIP analog